(19) United States
(12) Patent Application Publication
(10) Pub. No.: US 2024/0024323 A1
(43) Pub. Date: Jan. 25, 2024

(54) Methods of Administering Belumosudil for Treatment of Chronic Graft Versus Host Disease in Patient Subpopulations

(71) Applicant: Kadmon Corporation, LLC, Bridgewater, NJ (US)

(72) Inventor: Jeegar P. Patel, Hillsborough, NJ (US)

(73) Assignee: Kadmon Corporation, LLC, Bridgewater, NJ (US)

(21) Appl. No.: 18/105,372

(22) Filed: Feb. 3, 2023

Related U.S. Application Data
(63) Continuation of application No. PCT/US2022/037192, filed on Jul. 14, 2022.

(51) Int. Cl. A61K 31/517; A61P 37/06; A61K 9/20

(52) U.S. Cl. CPC A61K 31/517; A61P 37/06; A61K 9/2009; A61K 9/2013; A61K 9/2054; A61K 9/2027; A61K 9/2031

(57) ABSTRACT

The present disclosure provides methods of administering belumosudil mesylate salt to certain subpopulations of patients with cGVHD.

### TECHNICAL FIELD

The present disclosure relates to methods of administering belumosudil mesylate (REZUROCK™) for the treatment of chronic graft-versus-host disease (cGVHD) in certain subpopulations of patients.

### BACKGROUND

Chronic graft-versus-host disease (cGVHD) is an immune-mediated inflammatory and fibrotic disorder. It is a potential, serious complication following solid organ transplant and allogeneic hematopoietic cell transplant (alloHCT). cGVHD affects up to 70% of all alloHCT recipients, with an incidence of 20%-50% in children. It is the leading cause of non-relapse mortality beyond 2 years after alloHCT. The estimated prevalence of cGVHD is 14,000 patients in the United States (as of 2016). (Bachier C R et al: Epidemiology and real-world treatment of chronic graft-versus-host disease post allogeneic hematopoietic cell transplantation: A US claims analysis. Presented at ASH 2019, Orlando, FL, Dec. 7-10, 2019) (“Bachier et al.”)

Patients with cGVHD have substantial impairment in quality of life (QOL) as assessed by the Lee Symptom Scale (LSS), which measures the effect of cGVHD on patients' functioning and well-being. It is reported that only one third of patients who have cGVHD and start systemic treatment will be alive, in remission and off immunosuppressive therapy by 5 years. (Lee S J et al: Success of immunosuppressive treatments inpatients with chronic graft-versus-host disease. Biol Blood Marrow Transpl 24:555-562, 2018) (“Lee et al.”).

The pathophysiology of cGVHD can be separated into three phases: early inflammation because of tissue injury, a dysregulated adaptive immune system, and chronic inflammation and aberrant tissue repair with fibrosis.

First-line therapy for National Institutes of Health (NIH)-defined moderate to severe chronic graft-versus-host disease (cGVHD) is corticosteroids alone or in combination with sirolimus or a calcineurin inhibitor. However, up to 70% of patients require additional lines of therapy. (Bachier C R et al). Furthermore, the long-term use of corticosteroids is associated with significant side effects. (Lee et al).

Management of cGVHD continues to evolve with the advent of targeted therapies. cGVHD is characterized by an overproduction of proinflammatory cytokines IL-21 and IL-17, as well as overactivation of T follicular helper cells and B cells, which in turn leads to overproduction anti-bodies.

In 2017, the US Food and Drug Administration approved ibrutinib, a Bruton's Tyr kinase inhibitor, for the treatment of adults with cGVHD after failure of one or more 1 systemic lines of therapy. In patients with cGVHD who were required to have either >25% body surface area erythematous rash or an NIH mouth score of >4, a study with ibrutinib reported an overall response rate (ORR) of 67% and a discontinuation rate because of treatment-emergent adverse events (TEAEs) of 43%. (Waller E K, et al: Ibrutinib for chronic graft-versus-host disease after failure of prior therapy: 1-Year update of a phase 1b/2 study. Biol Blood Marrow Transpl 25:2002-2007, 2019).

There remains an opportunity to study other treatment options for patients with cGVHD, including those female patients who have reproductive potential and male patients with female partners of reproductive potential.

### SUMMARY

The present disclosure provides methods of administering belumosudil, in some embodiments, belumosudil mesylate (REZUROCK™) to certain at-risk subpopulations of patients for treatment of cGVHD.

In one embodiment, the present disclosure provides 2-{3-[4-(1H-indazol-5-ylamino)-2-quinazolinyl]phenoxy}-N-(propan-2-yl) acetamide, or a pharmaceutically acceptable salt thereof, (Compound) for use in the treatment of cGVH-ID in a male or female patient of reproductive potential, comprising the step of advising the patient to use effective contraception during treatment and for at least one week after the last dose of Compound.

In another embodiment, the present disclosure provides for use of 2-{3-[4-(1H-indazol-5-ylamino)-2-quinazolinyl]phenoxy}-N-(propan-2-yl)acetamide, or a pharmaceutically acceptable salt thereof (Compound), in some embodiments, the mesylate salt thereof (Belumosudil), in the treatment of a female patient of reproductive potential with cGVHD, comprising the step of verifying the pregnancy status of the patient prior to initiating treatment with Compound.

In another embodiment, the present disclosure provides for use of Belumosudil in the treatment of chronic graft-versus-host disease (cGVHD) in a patient who is lactating comprising advising the patient not to breastfeed during treatment with Compound and for at least one week after the last dose of Compound.

In other embodiment, the disclosure provides for use of in the treatment of chronic graft-versus-host disease (cGVHD) in a pregnancy-risk patient comprising the step of advising the patient of reproductive risks associated with treatment with Compound.

In another embodiment, the present disclosure relates to methods of treating a patient with cGVHD, comprising a step of verifying, prior to treatment, whether the patient is a reproductive risk patient. The disclosure further provides for administering Compound to the patient if it is verified that the patient is not a reproductive risk patient; or alternatively, if it is verified that the patient is a reproductive risk patient, advising the patient of potential reproductive risks in receiving treatment with Belumosudil; and/or using or advising thtef use of effective contraception during treatment with Belumosudil and for at least one week after receiving the last dose thereof.

The present embodiments can be understood more fully by reference to the detailed description and examples, which are intended to exemplify non-limiting embodiments.

### DETAILED DESCRIPTION

### Overview

Belumosudil is an oral selective rho-associated coiled-coil-containing protein kinase-2 (ROCK2) inhibitor. ROCK2 inhibition acts on the dysregulated adaptive immune system and the fibrosis that occurs because of aberrant tissue repair. Belumosudil inhibits ROCK2 and ROCK1 with IC₅₀ values of approximately 100 nM and 3 μM, respectively.

Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. By controlling ROCK2 activity, belumosudil mediates signaling in immune cellular function and fibrotic pathways, thereby alleviating the effects caused by this debilitating disease, such as inflammation of multiple tissues and fibrotic changes that may involve several organs including the lungs, hepatobiliary system, musculoskeletal system, gastrointestinal (GI) tract, and skin.

The mesylate salt of belumosudil is marketed as REZUROCK™ in the United States and other countries for the treatment of patients with chronic GVHD (cGVHD), in some instances after failure of at least two prior lines of systemic therapy. The compound belumosudil has the chemical name: 2-{3-[4-(1H-indazol-5-ylamino)-2-quinazolinyl]phenoxy}-N-(propan-2-yl) acetamide. The compound belumosudil is also known as KD025. The active pharmaceutical ingredient of REZUROCK™ is belumosudil mesylate salt with the molecular formula C₂₇H₂₈N₆O₅S, a molecular weight of 548.62 g/mol, and having the chemical name 2-{3-[4-(1H-indazol-5-ylamino)-2-quinazolinyl]phenoxy}-N-(propan-2-yl) acetamide methanesulfonate (1:1).

The chemical structure of belumosudil mesylate is as follows:

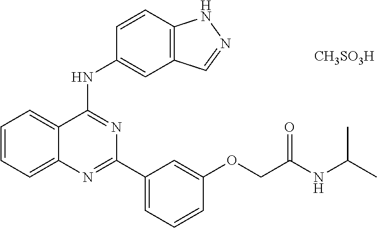 [SMILES: CC(C)NC(=O)COc1cccc(-c2nc(Nc3ccc4[nH]ncc4c3)c3ccccc3n2)c1 ; "CH3SO3H"]

Belumosudil and processes for making the compound are described in the following US patents: U.S. Pat. Nos. 8,357,693, 9,815,820, 10,183,931, and 10,696,660.

Applicant herein has evaluated belumosudil in multiple rat and rabbit embryo-fetal toxicology studies as described below in Examples 1-5 (i.e., Example 1, titled “A Pilot Prenatal Developmental Toxicity and Toxicokinetic Study in Sprague-Dawley Rats; Example 2, titled “A Pilot Prenatal Developmental Toxicity and Toxicokinetic Study in New Zealand White Rabbits With a Non-pregnant Dose Range Finding Phase”; Example 3, titled, “An Embryo-Fetal Developmental Toxicity Study in Sprague-Dawley Rats With a Toxicokinetic Evaluation”; Example 4, titled, “An Embryo-Fetal Developmental Toxicity Study in New Zealand White Rabbits With a Toxicokinetic Evaluation”; and Example 5, titled “A Combination Study of Fertility and Early Embryonic Development to Implantation in Sprague-Dawley Rats).

Based on findings from the aforesaid studies, belumosudil has the potential to cause fetal harm when administered to a pregnant woman. In the aforesaid animal studies, belumosudil was administered to pregnant rats during the period of organogenesis at oral doses of 25, 50, 150, and 300 mg/kg/day in a pilot study (Example 1 herein) and doses of 15, 50, and 150 mg/kg/day in a pivotal study (Example 3 herein). In the pilot study, maternal toxicity and embryo-fetal developmental effects were observed. Maternal toxicity (reduced body weight gain) occurred at 150 and 300 mg/kg/day doses. Increased post-implantation loss occurred at 50 and 300 mg/kg/day. Fetal malformations were observed at ≥50 mg/kg/day and included absence of anus and tail, omphalocele, and dome shaped head. The exposure (AUC) at 50 mg/kg/day in rats was approximately 3 times the human exposure at the recommended human dose of 200 mg.

In an embryo-fetal developmental study in rabbits (Example 4 herein), pregnant animals were administered oral doses of belumosudil at 50, 125, and 225 mg/kg/day during the period of organogenesis which resulted in maternal toxicity and embryo-fetal developmental effects. Maternal toxicity (body weight loss and mortality) was observed at doses ≥125 mg/kg/day. Embryo-fetal effects were observed at doses ≥50 mg/kg/day and included spontaneous abortion, increased post-implantation loss, decreased percentage of live fetuses, malformations, and decreased fetal body weight. Malformations included those in the tail (short), ribs (branched, fused or deformed), sternebrae (fused), and neural arches (fused, misaligned, and deformed). The exposure (AUC) at 50 mg/kg/day in rabbits was approximately 0.07 times the human exposure at the recommended dose of 200 mg.

In a combined male and female rat fertility study (Example 5 herein), belumosudil-treated male animals were mated with untreated females, or untreated males were mated with belumosudil-treated females. Belumosudil was administered orally at doses of 50, 150 or 275 mg/kg/day to male rats 70 days prior to and throughout the mating period, and to female rats 14 days prior to mating and up to Gestation Day 7. At the dose of 275 mg/kg/day, adverse findings in female rats (treated with belumosudil or untreated but mated with treated males) included increased pre-or post-implantation loss and decreased number of viable embryos. Administration of belumosudil to male rats at a dose of 275 mg/kg/day resulted in abnormal sperm findings (reduced motility, reduced count, and increased percentage of abnormal sperm), and testes/epididymis organ changes (reduced weight and degeneration). Fertility was reduced in both treated males or females at the 275 mg/kg/day dose and reached statistical significance in males. Adverse changes in male and female reproductive organs also occurred in general toxicology studies; findings included spermatozoa degeneration at a belumosudil dose of 35 mg/kg/day in dogs and decreased follicular development in ovaries at 275 mg/kg/day in rats. Changes were partially or fully reversed during the recovery period. The exposure (AUC) at the doses of 35 mg/kg/day in dogs, and 275 mg/kg/day in rats is 0.5 times and 8-9 times, respectively, the clinical exposure at the recommended dose of 200 mg daily.

The present disclosure provides methods of administering belumosudil, in some embodiments, belumosudil mesylate (REZUROCK™) to certain subpopulations of patients including females with reproductive potential and pregnancy risk partners. Because of the potential for adverse reactions in a child being breastfed by a patient who is taking Belumosudil, this subpopulation also includes lactating women.

### Definitions

“About” as used herein includes the exact amount modified by the term, about, as wells as an amount that would be expected to be within experimental error, such as for example, within 15%, 10%, or 5%. For example, “about 200 mg” means “200 mg” and also a range of mgs that is within experimental error, e.g., plus or minus 15%, 10%, or 5% of 200 mg. As used herein, the term “about” may be used to modify a range and also, a particular value.

“Administering” or “administered to” as used herein (for example, with reference to administration of API, including Compound or belumosudil, to a subject), refers to the act of prescribing medicine(s) containing the API for the subject to take during treatment, the act of dispensing the medicine(s) to the subject, and/or the act of physically receiving or ingesting the medicine(s). Thus, the API (e.g., Compound or belumosudil), can be “administered” by a physician or other medical professional who writes prescriptions for medicine(s); and/or by a pharmacist who fills said prescriptions and/or dispenses the medicine(s) to the subject; and/or by the patient or subject who ingests the medicine and/or his or her partner or caretaker.

“API” means “active pharmaceutical ingredient.”

“Allogeneic hematopoietic stem cell transplantation (allo-HSCT)” also called bone marrow transplantation or stem cell transplantation, or “allogeneic hematopoietic cell transplantation (allo-HCT)” refers to a procedure where hematopoietic cells from a donor are grafted into a recipient who is not an identical twin. The source of hematopoietic stem cells for allogeneic transplantation may be peripheral blood stem cells (PBSC) or bone marrow (BM). In some circumstances umbilical cord blood may be used. The donor and recipient may be matched at the human leukocyte antigen (HLA) genes, such as siblings. The donor and recipient may be a parent and a child who are only half-matched (haploidentical).

“Belumosudil” as used refers to the compound belumosudil in any form as well as pharmaceutically acceptable salts thereof, unless the context clearly indicates otherwise. The term “belumosudil” refers both to the compound belumosudil (for example, in the free base form, amorphous form, or crystalline form), to pharmaceutically acceptable salts of belumosudil, for example, the mesylate salt form as used in as REZUROCK,™ and to any form of belumosudil that may be used in a formulation or pharmaceutical composition for administering the compound to a patient.

“Breastfeed” means delivering milk to a child that is produced through lactation and includes both administering the milk to a child from the breast and/or collecting the breastmilk and delivering it by other means, for example, from bottles or containers.

“Clinical endpoint” or “study endpoint” refers to an event or outcome in a clinical trial that can be measured objectively to determine outcomes and potential beneficial effects of the drug or administration protocol as designed in the clinical trial. Examples of clinical endpoints include the following. Overall response rate (ORR) is the percentage of people in a study or treatment group who have a partial response (PR) or complete response (CR) to the treatment within a certain period of time. Failure-free survival (FFS) means the time from the first dose of belumosudil to a failure event, or the interval between the start of belumosudil and the addition of a new cGVHD therapy, relapse of the underlying disease, or nonrelapse mortality (NRM). Overall survival (OS) means the length of time from either the date of diagnosis or the start of treatment for a disease. Duration of response (DOR) means from the time of initial response (e.g., PR or CR) until documented progression from best response of cGVHD, time from initial response to start of additional systemic cGVHD therapy, or death. Time to next treatment (TTNT) means time to initiation of a subsequent systemic cGVHD therapy.

“Clinically recommended amount” or “clinically recommended dosage” refers to the amount or dosage of API that has been recommended and/or approved for administration to a patient by those skilled in the field of medicinal chemistry to treat the disease state in question following clinical trials. In some embodiments, the clinically recommended amount of Belumosudil is 200 mg once daily taken with food until progression of chronic GVHD that requires new systemic therapy.

“Compound” as used in the Claims and Embodiments herein, and when apparent from context of usage, is synonymous with the above all-inclusive definition of Belumosudil.

“CYP3A” refers to the CYP3A family of p-450 isoenzymes including CYP3A4.

“Effective contraception” is the use of artificial methods or other techniques to effectively prevent pregnancy from sexual intercourse. Examples of contraception include barrier methods, e.g., the condom; hormonal methods (e.g., the contraceptive pill); intrauterine devices, such as the coil; and male or female sterilization. Simultaneous use of multiple forms of contraception are contemplated by the definition of effective contraception and increase the effectiveness of the contraception.

“Fetus” as used herein encompasses an unborn offspring in the process of development, including a fertilized egg, embryo, or fetus in later stages of development.

“Gestation” means the period of intrauterine development from conception to birth

“Immunosuppressive therapy” (IST) refers to therapy that is typically administered for at least six months after allo-HSCT to try to prevent GVHD. Examples of IST's include sirolimus, prednisone and calcineurin inhibitors such as tacrolimus and cyclosporine.

“Lactating patient” means a patient who is capable of producing breastmilk.

Lee Symptom Scale (LSS) summary score measures the effect on patients' functioning and well-being. The Lee Symptom Scale is a 30-item scale developed to measure the symptoms of cGVHD and is described in Lee S J, et al., Development and validation of a scale to measure symptoms of chronic graft-versus host disease. Biol Blood Marrow Transplant 2002; 8:444-452.

“Line of treatment” or “line of therapy” describes the sequence or order in which different therapies are given to a patient as the patient's disease progresses. Initial treatment (first-line therapy) may not work or may stop working after a period. After first-line therapy is discontinued, a second different treatment (second-line therapy) may be given. Subsequent lines of therapy may be given when a second-line therapy does not work or stops working. Some patients may be administered multiple lines of therapy over the course of a disease.

First-line therapy for National Institutes of Health (NIH)-defined moderate to severe chronic graft-versus-host disease (cGVHD) may be corticosteroids alone or in combination with sirolimus or a calcineurin inhibitor. (Carpenter P A, et al.: A phase II/III randomized, multicenter trial of prednisone sirolimus versus prednisone sirolimus calcineurin inhibitor for the treatment of chronic graft-versus-host disease: BMT CTN 0801. Haematologica 103:1915-1924, 2018).

Examples of corticosteroid therapies for treatment of cGVHD include, but are not limited to, prednisone, prednisolone, methylprednisolone, and budesonide. Examples of prior systemic therapies for treating cGVHD include, but are not limited to, prednisone, tacrolimus, extracorporeal photopheresis (ECP), sirolimus, ibruitinib, ruxolitinib, mycophenolate mofetil (MMF), rituximab, methotrexate (MTX), cyclosporine, imatinib, ixazomib, and ofatumumab.

“Malformation” is a permanent deviation which generally is incompatible with or severely detrimental to normal postnatal survival or development. “Malformation” means a structural anomaly that alters general body conformity, disrupts or interferes with body function, or is generally thought to be incompatible with life. Specific examples of processes that result in malformation include marked or severe misshaping, asymmetry or irregularity of structure brought about by fusion, splitting, disarticulation, malalignment, hiatus, enlargement, lengthening, thickening, thinning, or branching. Absence of parts or whole structures is also considered a malformation.

“Myeloablative transplant” refers to a transplantation process using very high doses of chemotherapy or radiation prior to transplantation with autologous or allogeneic hematopoietic stem cells. A non-myeloablative transplant, or reduced intensity transplant, involves the patient having less intensive chemotherapy before transplantation with allogeneic hematopoietic stem cells.

“NIH lung symptom score” or “NIH cGVHD lung score” is a clinical symptom-based score ranging from 0 to 3. A Score 0 is used for no symptoms, Score 1 is used for symptoms of shortness of breath with stairs, Score 2 is used for symptoms of shortness of breath on flat ground, and Score 3 is used for shortness of breath at rest or requiring oxygen.

“Or” is used in the inclusive sense (equivalent to “and/or”) unless the context requires otherwise.

“Patient” as used herein includes an animal or a human, and in one embodiment, a human, who is in need of, or a candidate for, treatment with Belumosudil or receiving treatment with Belumosudil.

“Pregnancy” or “pregnant” as used herein means the state where a female patient has a fertilized egg, embryo or fetus developing in her uterus and encompasses all stages of fetal development from time of conception through birth.

“Pregnancy-risk patient” as used herein means any patient whose age, gender, and/or life circumstances poses a risk of pregnancy in herself or a risk of creating pregnancy in another. Thus, a “pregnancy-risk patient” includes a female patient of reproductive potential, as well as a male patient who is sexually active with a female(s) of reproductive potential. The term “pregnancy-risk patient” does not include a pregnant patient.

“Pregnancy risk partner” as used herein means any person who has a relationship with a pregnancy-risk patient that may create a risk of pregnancy in the patient, by any means (e.g., via natural means or in vitro fertilization).

“Reproductive potential” when used with reference to a female patient means the patient is physiologically able to produce egg cells (ova or oocytes), and/or capable of carrying a fetus in utero (e.g., once implanted via artificial insemination). A female patient of “reproductive potential” does not include a female who is confirmed or verified (e.g., via laboratory testing), as pregnant. “Reproductive potential” when used with reference to a male patient means a male who is capable of producing sperm to fertilize a female egg cell.

“Reproductive risks” include a risk of adverse impact to fetal or embryonic development including, for example, reduced body weight, injuries, and/or malformations, as well as fertility risks and risks to children fed with breastmilk obtained from patients in treatment.

“Reproductive risk patient” means a patient who is capable of posing or creating a reproductive risk as defined herein, including a female patient of reproductive potential, a male patient of reproductive potential, a lactating patient or a pregnant patient.

“Risk” as used herein, for example, in the definitions of “pregnancy-risk patient” and “pregnancy-risk partner” means there is a possibility, even if slight or remote, of a pregnancy taking place.

“Side effect” means a physiological response attributable to a treatment other than desired effects. In certain embodiments, side effects may include embryo-fetal developmental effects and malformations. Side effects may be detected directly or indirectly.

“Steroid-refractory” (SR) cGVHD is defined as cGVHD progression while on steroids or corticosteroids; in one embodiment, while on prednisone.

“Subject” means an animal being treated with belumosudil including an animal or human subject.

A “therapeutically effective amount” of an API means an amount which, when administered to a human for treating a disease (for example, cGVHD), is sufficient to effect treatment for the disease state being treated. As applied to cGVHD in a human, “treating” or “treatment” includes (1) reducing the risk of developing cGVHD and/or inhibiting cGVHD, i.e., arresting or reducing the development of cGVHD or its clinical symptoms; and (2) relieving cGVHD, i.e., causing regression, reversal, or amelioration of the cGVHD or reducing the number, frequency, duration or severity of its clinical symptoms.

The therapeutically effective amount of an API may vary depending upon the health and physical condition of the subject to be treated, the extent of disease progression, the assessment of the medical situation, and other relevant factors. It is expected that the therapeutically effective amount may fall within a range that can be determined through trial and through reference to clinical trial data and results, for example, as described in Examples 1 and 2 hereof and in scientific literature.

“Verifying” as used herein with reference to the step of verifying the pregnancy status of a female subject includes any form or manner of investigation to ascertain the pregnancy status of a female subject including through physical examination, consultation, and/or diagnostic test(s). It should be understood that a person may “verify” the pregnancy status of a female subject by conducting a consultation, conducting a physical examination, asking that diagnostic tests be performed, ordering or prescribing diagnostic tests, performing diagnostic pregnancy tests, and/or receiving the results of any such tests or examinations to confirm the pregnancy status of the female subject.

### Exemplary Embodiments

In one embodiment, the present disclosure provides 2-{3-[4-(1H-indazol-5-ylamino)-2-quinazolinyl]phenoxy}-N-(propan-2-yl) acetamide, or a pharmaceutically acceptable salt thereof, (Compound), or the mesylate salt thereof (Belumosudil), for use in the treatment of cGVHD in certain subpopulations of patients who are reproductive risk patients.

In one embodiment, the reproductive risk (RR) patient is a female patient of reproductive potential; in another embodiment, the RR patient is a male patient with a female partner of reproductive potential; in another embodiment, the RR patient is a lactating patient; and in another embodiment, the RR patient is a pregnant patient.

In some embodiments, the RR patient is a female patient of reproductive potential using effective contraception during treatment; in some embodiments, said female patient is using effective contraception during treatment and for at least one week after the last dose of Compound; in some embodiments, the RR patient is a male patient of reproductive potential using effective contraception during treatment; in some embodiments, said male patient is using effective contraception during treatment and for at least one week after the last dose of Compound;

In one embodiment, the disclosure provides for use of Compound, or the mesylate salt thereof (Belumosudil), in the treatment of cGVHD in an RR patient, comprising the step of advising the patient of the reproductive risks to a fetus or embryo, and/or to a breastfed child, from treatment with Compound or Belumosudil. In another embodiment, the disclosure provides for verifying the status of the RR patient (for example, in the case of a female, whether she is pregnant), prior to administering the Compound, or Belumosudil.

In another embodiment, the disclosure provides for use of Compound, or the mesylate salt thereof (Belumosudil), in the treatment of cGVHD in a female patient of reproductive potential comprising advising the patient to use effective contraception during treatment and for at least one week after the last dose of Compound.

In another embodiment, the disclosure provides for use of Compound, or the mesylate salt thereof (Belumosudil), in the treatment of cGVHD in a female patient who is pregnant or becomes pregnant while taking Compound, comprising advising the female patient of the potential risk to a fetus in receiving treatment with Compound while pregnant.

In some embodiments, the patient being treated for cGVHD may become pregnant while receiving treatment with Compound. In said embodiments, the present disclosure contemplates advising the female patient of the potential risk to a fetus in receiving treatment with Compound while pregnant.

In some embodiments, the present disclosure provides for use of Compound, or the mesylate salt thereof (Belumosudil), in the treatment of cGVHD in a male patient having a female partner of reproductive potential comprising the step of advising the male patient to use effective contraception during treatment with Compound and for at least one week after the last dose of Compound.

In another embodiment, the present disclosure provides for use of Compound, or the mesylate salt thereof (Belumosudil), in the treatment of cGVHD in a patient who is lactating comprising the step of advising the patient not to breastfeed during treatment with Compound and for at least one week after the last dose of Compound.

In another embodiment, the present disclosure provides for use of Compound, or the mesylate salt thereof (Belumosudil), in the treatment of cGVHD in a pregnancy risk patient who is using effective contraception during treatment with Compound or Belumosudil. In some embodiments, the pregnancy risk patient is female; in other embodiments, the pregnancy risk patient is male. In another embodiment, the pregnancy risk patient is using effective contraception during treatment and for at least one week after the last dose of Compound.

In another embodiment, the present disclosure provides for use of Compound, or the mesylate salt thereof (Belumosudil), in the treatment of cGVHD in a patient who is not lactacting.

In some embodiments, the Compound, or the mesylate salt thereof (Belumosudil), is administered to the patient at a dose of 200 mg daily.

The present disclosure further provides a method of treating a patient for cGVHD, with Compound, or the mesylate salt thereof (Belumosudil), comprising the steps of: (a) verifying whether the patient is a reproductive risk patient; and (b) (i) if it is verified that the patient is not a reproductive risk patient, administering Belumosudil to the patient, or (ii) if it is verified that the patient is a reproductive risk patient, advising the patient of potential reproductive risks in receiving treatment with Belumosudil, and/or to use effective contraception during treatment with Compound and for at least one week after the last dose of Compound; and/or in the case of a lactating patient, not to breastfeed during treatment with Compound and for at least one week after the last dose of Compound.

In some embodiments, it may be verified that the patient is a lactating patient; or a pregnant patient; or a male or female pregnancy risk patient. In said embodiments, the present disclosure contemplates advising the patient of reproductive risks from Belumosudil; and/or to use effective contraception during treatment with Compound and for at least one week after the last dose of Compound; and/or where applicable, not to breastfeed, during treatment with Belumosudil and for at least one week after receiving the last dose thereof.

In some embodiments, the disclosure provides a method of treating cGVHD in a patient comprising: (a) verifying whether the patient is a reproductive risk patient; and (b)(i) if it is verified that the patient is not a reproductive risk patient, administering Belumosudil to the patient, or (ii) if it is verified that the patient is a reproductive risk patient, using effective contraception during treatment with Belumosudil and for at least one week after receiving the last dose.

In some embodiments, the subject (or patient) has had allogeneic hematopoietic stem cell transplantation that is a matched-HSCT. In some embodiments, the allogeneic hematopoietic stem cell transplantation is a haploidentical-HSCT.

In some embodiments, the belumosudil treatment is continued based on the patient's tolerability until active cGVHD symptoms resolve or progress. The number of cycles and duration of the treatment is patient dependent. In some embodiments, the belumosudil is administered to the patient in one or more 28-day cycles.

In some embodiments, the number of cycles range from 3 to 15. In some embodiments, the number of cycles range from 3 to 14, from 3 to 13, from 3 to 12, from 3 to 11, from 3 to 10, from 3 to 9, from 3 to 8, from 3 to 7, from 3 to 6, from 3 to 5, or from 3 to 4. In some embodiments, the number of cycles ranges from 5 to 11. In some embodiments, the number of cycles ranges from 6 to 12. In some embodiments, the number of cycles ranges from 5 to 10, from 5 to 9, or from 5 to 8. In some embodiments, the number of cycles ranges from 5 to 7. In some embodiments, the number of cycles ranges from 5 to 6. In some embodiments, the number of cycles is 5. In some embodiments, the number of cycles is 6. In some embodiments, the number of cycles is 7. In some embodiments, the number of cycles is 3, 4, 5, 6, 7, 8, 9, 10, 11, 12, 13, 14, or 15.

In some embodiments, the subject has chronic graft-versus-host disease and has failed one to three prior lines of systemic therapy for the chronic graft-versus-host disease. In some embodiments, the subject has chronic graft-versus-host disease and has failed at least two prior lines of systemic therapy for the chronic graft-versus-host disease. In some embodiments, the subject has chronic graft-versus-host disease and has failed two to five prior lines of systemic therapy for the chronic graft-versus-host disease. In some embodiments, the subject has failed at least one, at least two, at least three, at least four, or at least five.

In some embodiments, the subject experienced a complete response to last treatment for the graft-versus-host disease prior to belumosudil. In some embodiments, the subject experienced a partial response to last treatment for the graft-versus-host disease prior to belumosudil. In some embodiments, stable disease during the last treatment for the graft-versus-host disease prior to belumosudil.

In some embodiments, the prior lines of systemic therapy for the chronic graft-versus-host disease have been discontinued.

In some embodiments, the prior lines of systemic therapy are selected from the group consisting of prednisone, tacrolimus, ECP, sirolimus, ibruitinib, ruxolitinib, MMF, rituximab, MTX, cyclosporine, imatinib, ixazomib, and ofatumumab.

In some embodiments, the cGVHD is steroid-refractory (SR) cGVHD. In some embodiments, the subject is refractory to the last line of treatment prior to belumosudil treatment.

In some embodiments, the subject is receiving concomitant corticosteroid therapy. In some embodiments, the concomitant corticosteroid therapy is selected from the group consisting of prednisone, prednisolone, methylprednisolone, and budesonide. In some embodiments, the concomitant corticosteroid therapy is prednisone. In some embodiments, the dose of the concomitant corticosteroid therapy is reduced after at least 1 cycle of the belumosudil treatment. In some embodiments, the dose of the concomitant corticosteroid therapy is reduced by at least about 10%, by at least about 20%, by at least about 30%, by at least about 40%, by at least about 50%, by at least about 60%, or by at least about 70% after at least 1 cycle of the belumosudil treatment. In some embodiments, the dose of the concomitant corticosteroid therapy is reduced by from about 10% to about 70%, from about 15% to about 65%, from about 20% to about 60%, from about 30% to about 60%, from about 35% to about 60%, from about 40% to about 60%, or from about 45% to about 55% after at least 1 cycle of the belumosudil treatment. In some embodiments, the concomitant corticosteroid therapy is discontinued after at least 1 cycle of the belumosudil treatment.

In some embodiments, the subject is receiving concomitant calcineurin inhibitor therapy.

### Belumosudil Tablets

In one embodiment, the belumosudil is formulated into a tablet for oral administration. Belumosudil mesylate is a yellow powder that is practically insoluble in water. Belumosudil tablets may be prepared for oral administration. Each tablet contains 200 mg of the free base equivalent to 242.5 mg of belumosudil mesylate. The tablet also may contain the following inactive ingredients: microcrystalline cellulose, hypromellose, croscarmellose sodium, colloidal silicon dioxide, and magnesium stearate. The tablet film consists of polyvinyl alcohol, polyethylene glycol, talc, titanium dioxide and yellow iron oxide. Each 200 mg tablet is a pale-yellow film-coated oblong tablet debossed with “KDM” on one side and “200” on the other side. Tablets are stored at room temperature, 20° C. to 25° C. (68° F. to 77° F.); excursions permitted from 15° C. and 30° C. (59° F. to 86° F.).

The following abbreviations may be helpful in considering the description herein.

### Abbreviations

| | |
|---|---|
| AE | Adverse events |
| AMS | Accelerator mass spectrometry |
| alloHCT | allogeneic hematopoietic cell transplantation |
| BID | Twice daily (bi-daily) |
| BM | Bone marrow |
| cGVHD | Chronic graft versus host disease |
| CMV | cytomegalovirus |
| CR | Complete response |
| DDI | Drug-drug interaction |
| DOR | Duration of response |
| EOI | End of infusion |
| FFS | Failure-free survival |
| GD | Gestation day |
| HLA | human leukocyte antigen |
| IST | Immunosuppressive therapy |
| IV | Intravenous |
| LSC | Liquid scintillation counting |
| LSS | Lee Symptom Scale |
| NMT | Not more than |
| NOAEL | No-Observed-Adverse-Effect Level |
| ORR | Overall response rate |
| OS | Overall survival |
| PBSC | peripheral blood stem cells |
| PR | Partial response |
| QOL | Quality of life |
| SD | Standard deviation |
| SR | Steroid refractory |
| TEAEs | treatment-emergent adverse events |
| TK | Toxicokinetic |
| TTNT | Time to next treatment |
| QD | Daily; every day |

### EXAMPLES

### Example 1: A Pilot Prenatal Developmental Toxicity and Toxicokinetic Study in Sprague-Dawley Rats

### Study Objectives and Design

This pilot study was conducted to provide information regarding dose selection of belumosudil for use in subsequent embryo-fetal developmental toxicity studies in Sprague-Dawley rats (Example 3). A second part of the study was a pilot developmental toxicity study, i.e., to determine the toxicokinetics of belumosudil and its two metabolites, KD025 m1 and KD025m2, when belumosudil was administered once daily to pregnant rats via oral gavage from Gestation Day (GD) 6 through 17. The design of the study is shown in Table 1.

TABLE 1
Design of Dose Pilot/Rat Study of Example 1

| | | | | | Number of Animals | | |
|---|---|---|---|---|---|---|---|
| | | | Dose | Dose | | Uterine | |
| | | Dose Level | Volume | Concentration | Initial | Exams | Toxicokinetic |
| Group | Treatment | (mg base/kg) | (mL/kg) | (mg base/mL) | F | F | F |
| 1 | Control | 0 | 10 | 0 | 5 | 5 | — |
| 2 | Low Dose | 25 | 10 | 2.5 | 5 | 5 | — |
| 3 | Low-Mid Dose | 50 | 10 | 5.0 | 5 | 5 | — |
| 4 | Mid-High-Dose | 150 | 10 | 15.0 | 5 | 5 | — |
| 5 | High Dose | 300 | 10 | 30.0 | 5 | 5 | — |
| 6 | Control | 0 | 10 | 0 | 3 | — | 3 |
| 7 | Low Dose | 25 | 10 | 2.5 | 6 | — | 6 |
| 8 | Low-Mid Dose | 50 | 10 | 5.0 | 6 | — | 6 |
| 9 | Mid-High-Dose | 150 | 10 | 15.0 | 6 | — | 6 |
| 10 | High Dose | 300 | 10 | 30.0 | 6 | — | 6 |

| | | | |
|---|---|---|---|
| Total Number of Animals: | 52 | 25 | 27 |

A total of 52 Sprague-Dawley (SD) rats were used in this study. A vehicle, 0.4% (w/w) methylcellulose (400 cps) in distilled water, or belumosudil, were administered to time-mated female SD rats once daily via oral gavage from Gestation Day (GD) 6 through 17 at approximately the same time each day, with a dose volume of 10 mL/kg, at the varied dosage levels shown in Table 1. Animals were assigned to groups as shown in Table 1.

### Observations

Observations of the animals included cageside assessments (twice daily), evaluation of clinical signs (daily from GD 6 through 20), body weights (at arrival—GD 2 or 3-and GD 6, 9, 12, 15, 18, and 20), food consumption (GD 6-20), and anatomical pathology including a uterine examination. Toxicokinetic assessment was conducted for the belumosudil and metabolites. All fetuses were given an external examination.

Clinical signs were limited to 300 mg base/kg/day and were of low incidence but were considered likely related to treatment. Clinical signs consisted of unkempt and/or thin appearance, and hunched posture.

Mean body weight gains and food consumption at 25 and 50 mg base/kg/day were slightly reduced (dose-related) from GD 6-9, GD 6-18, and GD 6-20. At 150 mg base/kg/day, mean body weights were reduced throughout gestation, a body weight loss occurred from GD 6-9, and reduced body weight gains were notable from GD 12-15, GD 6-18, and GD 6-20. Food consumption at 150 mg base/kg/day was correspondingly decreased during these same intervals. Increases in body weight gain and food consumption following treatment (GD 18-20) at 25, 50, and 150 mg base/kg/day, were considered a rebound/recovery effect. Mean body weight values, mean body weight gains, and food consumption, were adversely affected at every interval after the initiation of dosing on GD 6 at 300 mg base/kg/day.

No adverse maternal necropsy findings were noted in any treated group. Litters available for examination numbered 5 each in the 0, 25, 50, 150, and 300 mg base/kg/day groups. At dose levels of 25, 50, and 150 mg base/kg/day, all the females sustained a pregnancy to the scheduled day of necropsy on GD 20.

A dose-response was not apparent in the intrauterine data at 25, 50, and 150 mg base/kg/day, although the mean number of early resorptions was notably increased, and the mean number of viable fetuses was slightly decreased in the 50 mg base/kg/day group. Mean post-implantation loss (% per litter) was 4.41, 4.00, 11.62, 4.51, and 24.51 at 0, 25, 50, 150, and 300 mg base/kg/day, respectively. Mean viable litter sizes at these same dose levels were 12.8, 13.2, 11.6, 12.2, and 10.0, respectively.

The mean fetal body weights (female fetuses) at 0, 25, 50, 150, and 300 mg base/kg/day were 1.36, 4.19, 4.39, 4.18 and 3.34 g, respectively. The combined mean fetal body weights (males and females) at these same dose levels were 4.50, 4.37, 4.50, 4.22, and 3.42 g, respectively. Post implantation loss (early resorptions) was increased (including a female with total embryo loss), and mean fetal body weights were significantly decreased at 300 mg base/kg/day.

Although the historical prevalence of external fetal malformations in dose range-finding studies in rats is atypical, in this study the litter incidence was notable, ranging from 40% and 20% of the litters affected at 50 and 150 mg base/kg/day, respectively, to 25% of the litters affected at 300 mg base/kg/day. In the 50 mg base/kg/day group, one fetus was edematous (whole body) and the anus and tail were absent in another fetus/litter in this dose group. In the 150 mg base/kg/day group, one fetus number had omphalocele. One fetus at 300 mg base/kg/day had a dome-shaped head.

### Results

In this oral pilot prenatal developmental toxicity study with belumosudil, maternal toxicity was considered slight at 25 and 50 mg base/kg/day and moderate at 150 mg base/kg/day (with moderate defined as prominent with significant potential for increased severity. Limited tissue or organ function is possible.) Maternal, embryo, and fetal toxicity were considered excessive at 300 mg base/kg/day and would preclude this dose level for selection in a subsequent developmental toxicity study in rats.

For the parent compound, belumosudil, a dose level of 25 mg base/kg/day corresponded to a maternal Cₘₐₓ of 1800 ng/mL at the end of the dosing regimen (GD 17), and a maternal AUC₀₋₂₄ of 20400 ng·hr/mL; for KD025 m1, maternal Cₘₐₓ was 176 ng/mL and AUC₀₋₂₄ was 2230 ng·hr/mL, and for KD025m2, maternal Cₘₐₓ was 127 ng/mL and AUC₀₋₂₄ was 1480 ng·hr/mL.

### Example 2: A Pilot Prenatal Developmental Toxicity and Toxicokinetic Study in New Zealand White Rabbits With a Non-pregnant Dose Range Finding Phase

### Study Objectives and Design

This study was conducted to provide information for dose selection of belumosudil for use in subsequent embryo-fetal developmental toxicity studies in New Zealand White (NZW) rabbits (Example 4). The study was conducted in two phases: Phase A was a dose range finding tolerance study with non-pregnant rabbits, and Phase B was the pilot developmental toxicity study. The vehicle, 0.4% (w/w) methylcellulose (400 cps) in distilled water, or belumosudil, were administered to non-pregnant or time-mated female NZW rabbits once daily via oral gavage for two sets of 5 consecutive days (Phase A) or once daily via oral gavage from Gestation Day (GD) 6 through 18 (Phase B). The design of the study is further set forth in Table 2 below.

TABLE 2
Design of Dose Pilot/Rabbit Study of Example 2
Table 2: Study Design

| | | |
|---|---|---|
| Group | Dose Level | Number of |
| Number | (mg base/kg/day) | Females |

Phase A (Non-pregnant)^{a}

| | | |
|---|---|---|
| 1/5 | 25/100 | 2 |
| 2/6 | 50/200 | 2 |
| 3/7 | 75/300^{b} | 2^{b} |
| 4/8 | 125/400 | 2 |

Phase B Main Study (Time-mated)

| | | |
|---|---|---|
| 9 | 0 | 6 |
| 10 | 25 | 6 |
| 11 | 50 | 6 |
| 12 | 100 | 6 |
| 13 | 250 | 6 |

Phase B Toxicokinetic (Time-mated)

| | | |
|---|---|---|
| 14 | 0 | 4 |
| 15 | 25 | 4 |
| 16 | 50 | 4 |
| 17 | 100 | 4 |
| 18 | 250 | 4 |

^{a}Animals were administered the test article for 5 consecutive days followed by a 10-day washout period before being administered the test article at a higher dose level for 5 consecutive days;
^{b}the dose level is presented as a rising dose; however, one female (animal number 105) was replaced following the first dose. As a result, the replacement animal was only administered the second dose (300 mg base/kg/day); the total number of animals on study in Phase A was 9.

### Observations

Observations of the animals included clinical signs, body weights, food consumption, and anatomical pathology including a uterine examination. Toxicokinetic assessment was conducted for belumosudil and two metabolites. All fetuses were given the appropriate external examination.

Survival was not affected at any of the dose levels administered in Phase A. All the animals survived to the scheduled day of termination. Definitive treatment-related effects in Phase A were manifested at dose levels of 300 and 400 mg base/kg/day and consisted of slight body weight losses and decreased food consumption.

Potential treatment-related clinical findings seen at respective dose levels of 100, 200, 300 and 400 mg base/kg/day, administered from Study Days 15 through 19, were fecal abnormalities (few/absent) with brown discoloration in the anogenital region on the last day of the study (Study Day 20) for female number 108 at 400 mg base/kg/day.

Survival was not affected in Phase B at any of the doses administered. A dose-dependent maternal response occurred at dose levels of 100 and 250 mg base/kg/day and consisted of reduced body weight gain/loss, decreased food consumption, clinical signs (inappetence, fecal abnormalities, thin appearance), and a generally dose-related increase in the amount of veterinary intervention (food enrichment). No treatment-related maternal necropsy findings were observed, and no treatment-related embryo or fetal toxicity was apparent at any dose level.

### Results

When belumosudil was administered to pregnant rabbits from Gestation Days 6 through 18 at 25, 50, 100 and 250 mg base/kg/day, no toxicologically relevant maternal toxicity was observed at 25 or 50 mg base/kg/day. A dose-dependent maternal response at 100 and 250 mg base/kg/day was apparent by reduced body weight gain/loss, decreased food consumption, clinical signs (inappetence, fecal abnormalities, thin appearance), and a generally dose-related increase in the amount of veterinary intervention (food enrichment).

At the NOAEL for maternal toxicity (50 mg base/kg/day), for belumosudil, a dose level of 50 mg base/kg/day corresponded to a maternal Cₘₐₓ of 621 ng/mL at the end of the dosing regimen (GD 18), and a maternal AUC₀₋₂₄ of 2480 ng·hr/mL; for KD025 m1, maternal Cₘₐₓ was 528 ng/mL and AUC₀₋₂₄ was 1320 ng·hr/mL, and for KD025m2, maternal Cₘₐₓ was 1090 ng/mL and AUC₀₋₂₄ was 2990 ng·hr/mL. At the NOAEL for developmental toxicity (250 mg base/kg/day), for belumosudil, a dose level of 250 mg base/kg/day corresponded to a maternal Cₘₐₓ of 2100 ng/mL at the end of the dosing regimen (GD 18), and a maternal AUC₀₋₂₄ of 15600 ng·hr/mL; for KD025 m1, maternal Cₘₐₓ was 1040 ng/mL and AUC₀₋₂₄ was 4760 ng hr/mL, and for KD025m2, maternal Cₘₐₓ was 3080 ng/mL and AUC₀₋₂₄ was 19700 ng hr/mL.

Based on the results of this study, dose levels of 0, 50, 125, and 225 mg base/kg/day were selected for the embryo-fetal developmental toxicity study in New Zealand White rabbits (Example 4).

### Example 3: An Embryo-Fetal Developmental Toxicity Study in Sprague-Dawley Rats With a Toxicokinetic Evaluation

### Study Objectives and Design

This study was conducted to determine the embryo-fetal developmental toxicity, including the teratogenic potential, of belumosudil in Sprague-Dawley rats. This study also included a toxicokinetic (TK) evaluation to determine the exposure/toxicity relationship for belumosudil and its metabolites (KD025 m1 and KD025m2). The vehicle, 0.4% (w/w) methylcellulose (400 cps) in distilled water, or belumosudil were administered to time-mated SD rats once daily via oral gavage from Gestation Day (GD) 6 through 17.

For this study, a total of 140 time-mated female rats (approximately 8 to 10 weeks of age) were obtained from Charles River Laboratories, Raleigh, North Carolina, and acclimated from the time of arrival until the time of dosing on GD 6. During the acclimation period, the animals were observed twice daily with respect to general health and any signs of disease. All animals were given a detailed clinical examination and body weights were recorded prior to selection. Animals assigned to study had body weights within +20% of the mean body weight. Food consumption was collected during the acclimation period.

Using a standard, by weight, measured value randomization procedure, 133 female animals (weighing 162 to 226 g, at randomization) were assigned to the control, treatment, and TK groups as shown in Table 3.

TABLE 3
Group Assignments for Example 3 Study
Table 3: Group Assignments

| | | |
|---|---|---|
| Group | Dose Level | Number of Time-mated |
| Number | (mg base/kg/day) | Females |

Main Study

| | | |
|---|---|---|
| 1 | 0 | 25 |
| 2 | 15 | 25 |
| 3 | 50 | 25 |
| 4 | 150 | 25^{a} |

Toxicokinetic

| | | |
|---|---|---|
| 5 | 0 | 3 |
| 6 | 15 | 10 |
| 7 | 50 | 10 |
| 8 | 150 | 10 |

^{a}Five animals at 150 mg base/kg/day (animal numbers 277 and 289 to 292) were excluded from the summary tables due to suspected gavage injuries.

### Administration

The vehicle and belumosudil were administered once daily from GD 6 to 17 at approximately the same time each day (±2 hours from the GD 6 dose) via oral gavage. The dose levels for the treated groups were 15, 50, and 150 mg base/kg/day at a dose volume of 10 mL/kg. The control group received the vehicle in the same manner as the treated groups. Additionally, TK animals received the vehicle or belumosudil in the same manner as the main study groups at the same dose levels and volume. The vehicle and belumosudil formulations were continually stirred for at least 30 minutes at room temperature prior to and throughout dose administration. Individual doses were based on the most recent body weights.

### Observations

Animals received in-life and postmorten evaluations. In-life evaluations included cageside observations (for morbidity, mortality, injury), detailed clinical signs, body weights, food consumption, and anatomical pathology including a uterine examination. Toxicokinetic assessment was conducted for the belumosudil and metabolites (KD025 m1 and KD025m2). All fetuses were given an external and visceral or skeletal examination.

### Cageside and Clinical Observations.

No treatment related mortality was observed at any dose level. Five females at 150 mg base/kg/day sustained suspected gavage injuries. For clarity in discussing the study results, these five females were not included in tabulation of the summary data.

Detailed clinical observations were made daily from GD 6 through 20 (4 hours±1 hour postdose on dosing days). The observations included, but were not limited to, evaluation of the skin, fur, eyes, ears, nose, oral cavity, thorax, abdomen, external genitalia, limbs and feet, as well as evaluation of respiration. Clinical observations are summarized in Table 4.

TABLE 4
Summary of Gestation Clinical Observations
in Example 3 Study (Days 6 to 20)*

| | Dose (base/kg/day) | | | |
|---|---|---|---|---|
| Observation | 0 mg | 15 mg | 50 mg | 150 mg |
| Number of Animals Observed | 25 | 25 | 25 | 20 |
| External Appearance | | | | |
| Discharge, Brown, Vulva | 1/1 | 1/1 | 1/1 | 4/4 |
| Discharge, Red, Vulva | 1/1 | 0/0 | 4/4 | 1/1 |
| Ear/portion of ear missing, | 0/0 | 13/1 | 0/0 | 15/1 |
| Ear/left | | | | |
| Tail missing - portion | 0/0 | 0/0 | 0/0 | 11/1 |
| Pelage/Skin | | | | |
| Hair sparse, Abdominal region | 0/0 | 7/1 | 0/0 | 0/0 |
| Hair sparse, Cervical region | 0/0 | 0/0 | 8/1 | 0/0 |
| Hair sparse, Forefoot/left | 5/1 | 4/2 | 0/0 | 27/3 |
| Hair sparse, Forefoot/right | 0/0 | 0/0 | 0/0 | 23/3 |
| Hair sparse, Forelimb/left | 0/0 | 12/3 | 10/2 | 14/2 |
| Hair sparse, Forelimb/right | 0/0 | 17/3 | 10/2 | 30/3 |
| Hair sparse, Hind limb/left | 0/0 | 0/0 | 0/0 | 6/1 |
| Hair sparse, Hind limb/right | 0/0 | 0/0 | 0/0 | 6/1 |
| Hair sparse, Lumbar region | 0/0 | 0/0 | 0/0 | 12/1 |
| Hair sparse, Thoracic region | 0/0 | 0/0 | 7/1 | 12/2 |
| Scabbed area, Face | 0/0 | 7/1 | 0/0 | 0/0 |
| Scabbed area, Forelimb/right | 0/0 | 4/1 | 0/0 | 0/0 |
| Scabbed area, Tail | 0/0 | 0/0 | 0/0 | 13/2 |
| Scabbed area, Thoracic region | 0/0 | 1/1 | 0/0 | 0/0 |

*Number of times observed/Total number of animals

### Body Weights.

Body weights for all animals were measured and recorded on GD 6, 9, 12, 15, 18, and 20. Individual body weight change was calculated for the following GD intervals: 6-9, 9-12, 12-15, 15-18, 18-20, 6-18, and 6-20. Adjusted body weight (GD 20 body weight minus gravid uterine weight) and adjusted body weight change (GD 6 to 20) were also calculated.

Maternal body weights and body weight change are summarized below in Tables 5 and 6:

TABLE 5
Maternal Body Weights (g)

| Group (mg base/kg/day) | 0 | 15 | 50 | 150 |
|---|---|---|---|---|
| GD 9 | 236.4 | 231.7 | 225.5^{a} | 215.8^{b} |
| GD 12 | 262.3 | 257.0 | 246.3^{b} | 232.1^{b} |
| GD 15 | 279.3 | 272.8 | 262.6^{b} | 244.0^{b} |
| GD 18 | 320.7 | 315.5 | 298.1^{b} | 275.3^{b} |
| GD 20 | 355.8 | 349.6 | 335.2^{b} | 317.0^{b} |

^{a}Significantly different from control: p < 0.05
^{b}Significantly different from control: p < 0.01

TABLE 6
Maternal Body Weight Change (g)

| Group (mg base/kg/day) | 0 | 15 | 50 | 150 |
|---|---|---|---|---|
| GD 6-9 | 13.3 | 10.0 | 6.2^{b} | −4.2^{b} |
| GD 6-18 | 97.6 | 93.8 | 78.8^{b} | 55.4^{b} |
| GD 6-20 | 132.7 | 127.9 | 115.9^{b} | 97.1^{b} |
| GD 18-20 | 35.1 | 34.0 | 37.1 | 41.8^{b} |

^{S}Significantly different from control: p < 0.01

Mean maternal body weights were decreased with statistical significance in a dose-related manner at 50 and 150 mg base/kg/day throughout the treatment and post-treatment periods (GD 9, 12, 15, 18, and 20). Mean body weight gain was also reduced in a dose-related manner at 50 and

150 mg base/kg/day; the reduced body weight gains were statistically significant at 50 and 150 mg base/kg/day from GD 6-9 and 9-12. From GD 12-15, body weight gain at 50 mg base/kg/day was comparable to the control group, but remained significantly reduced at 150 mg base/kg/day during this same time period. Body weight gain remained somewhat decreased (statistically significant) at both dose levels during the last few days of the treatment period (GD 15-18), and following the treatment period (GD 18-20), mean body weight gain was either similar to (50 mg base/kg/day) or greater than (150 mg base/kg/day; statistically significant) that in the control group, indicating a recovery response. An evaluation of the comprehensive intervals (GD 6-18 and 6-20) revealed a consistent dose-related reduction in mean maternal body weight gain at 50 and 150 mg base/kg/day (statistically significant at both dose levels).

### Food Consumption.

Food consumption for main study animals was measured and recorded on the corresponding body weight days and calculated for the same intervals. Mean maternal food consumption was decreased with statistical significance in a dose-related manner at 50 and 150 mg base/kg/day throughout the treatment period (GD 6-9, 9-12, 12-15, 15-18, 6-18, and 6-20). Following the treatment period (GD 18-20), food consumption was similar to that in the control group, indicating a recovery response.

Maternal food consumption is summarized below in Table 7:

TABLE 7
Maternal Food Consumption/% Difference From Control

| Group (mg base/kg/day) | 0 | 15 | 50 | 150 |
|---|---|---|---|---|
| GD 6-9 | 20.3 | 19.0 | 15.5^{b} | 10.0^{b} |
| GD 6-18 | 23.7 | 22.6 | 20.0^{b} | 15.4^{b} |
| GD 6-20 | 24.3 | 23.2 | 21.3^{b} | 17.4^{b} |
| GD 18-20 | 27.9 | 27.3 | 29.0 | 29.4 |

^{b}Significantly different from control: p < 0.01

All the animals in the study were pregnant, resulting in a pregnancy index of 100% in each study group. Litters available for examination numbered 25, 25, 25, and 20 in the control, 15, 50, and 150 mg base/kg/day groups, respectively.

On GD 20, each surviving main study female was euthanized by carbon dioxide inhalation, followed by exsanguination of the abdominal vena cava and immediately subjected to a cesarean section. The skin was reflected from a ventral midline incision to examine mammary tissue and locate any subcutaneous masses. The abdominal cavity was then opened, and the uterus was exposed. The uterus was excised, and the gravid uterine weight was recorded. Beginning at the distal end of the left uterine horn, the location of viable and nonviable fetuses, early and late resorptions for each uterine horn, and the total number of implantations were recorded. The number of corpora lutea on each ovary was also recorded.

The fetuses were removed by making a dorsal incision longitudinally along both uterine horns. The embryonic membrane of each fetus was gently removed, and each fetus was pulled away from the placenta, fully extending the umbilical cord. The placentae were examined grossly.

Uteri from females that appeared nongravid were opened and placed in 10% ammonium sulfide solution for detection of implantation sites. The foci, if detected, were considered early resorptions, and data from this female were included in mean calculations

The mean gravid uterine weight was slightly reduced at 150 mg base/kg/day. The mean final body weight, adjusted final body weight, and adjusted final body weight change at 150 mg base/kg/day, however, were decreased with statistical significance when compared to the corresponding control values and were considered treatment-related.

The mean gravid uterine weight at 50 mg base/kg/day was similar to the control value. The mean final body weight, adjusted final body weight, and adjusted final body weight change at 50 mg base/kg/day, however, were decreased with statistical significance when compared to the corresponding control values and were considered treatment-related.

Gravid uterine weights and adjusted body weight/body weight changes are summarized in Table 8. Fetal body weights are summarized in Table 9.

TABLE 8
Summary of Gravid Uterine Weight and Adjusted
Body Weight/Body Weight Change Values

| | 0 mg | | 15 mg | | 50 mg | | 150 mg | |
|---|---|---|---|---|---|---|---|---|
| | base/kg/day | | base/kg/day | | base/kg/day | | base/kg/day | |
| | (N = 25) | | (N = 25) | | (N = 25) | | (N = 20) | |
| Endpoint | Mean | SD | Mean | SD | Mean | SD | Mean | SD |
| Gravid Uterine Weight, g | 69.3 | 9.15 | 71.0 | 9.59 | 68.8 | 8.42 | 64.3 | 10.54 |
| Final Body Weight, g | 355.8 | 24.12 | 349.6 | 22.81 | 335.2^{b} | 26.51 | 317.0^{b} | 21.49 |
| Adjusted Final Body | 286.5 | 19.52 | 278.5 | 19.88 | 266.4^{b} | 23.14 | 252.7^{b} | 15.85 |
| Weight, g | | | | | | | | |
| Adjusted Weight | 63.4 | 11.68 | 56.8 | 11.83 | 47.1^{b} | 10.64 | 32.8^{b} | 12.59 |
| Change From Day 6, | | | | | | | | |

N = Number of measures used to calculate mean
SD—Standard Deviation
^{b}= significantly different from control (p < 0.01)

TABLE 9
Summary of Mean Fetal Body Weights
(g)/% Difference From Control

| Group (mg base/kg/day) | 0 | 15 | 50 | 150 |
|---|---|---|---|---|
| Fetal Body Weight Males | 4.25 | 4.19 | 4.19 | 3.91^{b}/−8.0% |
| Fetal Body Weight Females | 4.03 | 4.00 | 3.94 | 3.72 ^{b}/−7.7% |
| Fetal Body Weight Genders | 4.12 | 4.10 | 4.06 | 3.82 ^{b}/−7.3% |
| Combined | | | | |

^{b} Significantly different from control: p < 0.01

For subjects treated at 15 and 50 mg base/kg/day, none of the fetal body weight values were statistically significant when compared to the respective control group values. All the mean fetal body weight values for males, females, and sexes combined, were decreased with statistical significance at 150 mg base/kg/day; the values were decreased between −7 and −8 compared to the corresponding control values.

### Toxicokinetic Analysis

Exposure to belumosudil, KD025 m1, and KD025m2 increased with the increase in belumosudil dose level from 15 to 150 mg base/kg. The increases in belumosudil Cₘₐₓ values for pregnant rats were generally less than dose proportional on GD 6 and GD 17 and generally dose proportional on GD 6 and GD 17 for AUC₀₋₂₄. The increases in KD025 m1 Cₘₐₓ values for pregnant rats were generally dose proportional on GD 6 and GD 17 and greater than dose proportional on GD 6 and GD 17 for AUC₀₋₂₄. The increases in KD025m2 Cₘₐₓ and AUC₀₋₂₄ values for pregnant rats were generally dose proportional on GD 6 and greater than dose proportional on GD 17. No apparent accumulation of belumosudil, KD025 m1, and KD025m2 was observed after multiple dosing of belumosudil in pregnant rats. The AUC₀₋₂₄ metabolite to parent ratios indicate that belumosudil is converted to KD025 m1 and KD025m2 in pregnant rats following oral gavage administration of belumosudil. Metabolite to Parent ratios ranged from 0.0799 to 0.188 and 0.0482 to 0.125 for KD025 m1 and KD025m2, respectively.

These TK results are summarized below in Table 10.

TABLE 10
Summary of the Mean Belumosudil, KD025m1, and KD025m2
Cₘₐₓ and AUC₀₋₂₄ in Plasma of Pregnant Rats

| Inter- | | | KD025 | | KD025m1 | | KD025m2 | |
|---|---|---|---|---|---|---|---|---|
| Val | Dose | Dose Level | Cₘₐₓ | AUC₀₋₂₄ | Cₘₐₓ | AUC₀₋₂₄ | Cₘₐₓ | AUC₀₋₂₄ |
| (GD) | Group | (mg base/kg) | (ng/mL) | (ng*hr/mL) | (ng/mL) | (ng*hr/mL) | (ng/mL) | (ng*hr/mL) |
| 6 | 6 | 15 | 1530 | 9750 | 152 | 978 | 76.2 | 515 |
| | 7 | 50 | 3000 | 37700 | 349 | 5040 | 191 | 3050 |
| | 8 | 150 | 5150 | 89400 | 955 | 16400 | 519 | 10500 |
| 17 | 6 | 15 | 1320 | 11900 | 93.3 | 948 | 55.6 | 572 |
| | 7 | 50 | 4360 | 33300 | 434 | 4780 | 350 | 3580 |
| | 8 | 150 | 7800 | 88300 | 1310 | 16600 | 972 | 11100 |

### Results

When belumosudil was administered to pregnant rats from Gestation Days 6 through 17 at 15, 50, and 150 mg base/kg/day, maternal and fetal developmental toxicity occurred at 150 mg base/kg/day; reductions in mean maternal body weights, body weight gains, and food consumption were significant. Mean fetal body weight also was significantly reduced and considered adverse. A dose level of 50 mg base/kg/day resulted in adverse dose-related reductions in mean maternal body weights, body weight gains, and food consumption. A dose level of 15 mg base/kg/day did not produce any adverse maternal or fetal developmental effects. A dose level of 50 mg base/kg/day was considered the NOAEL for fetal developmental toxicity.

For the parent compound, belumosudil, a dose level of 50 mg base/kg/day corresponded to a maternal Cₘₐₓ of 4360 ng/mL at the end of the dosing regimen (GD 17), and a maternal AUC₀₋₂₄ of 33300 ng·hr/mL; for KD025 m1, maternal Cₘₐₓ was 434 ng/mL and AUC₀₋₂₄ was 4780 ng·hr/mL, and for KD025m2, maternal Cₘₐₓ was 350 ng/mL and AUC₀₋₂₄ was 3580 ng hr/mL. A dose level of 15 mg base/kg/day was considered the NOAEL for maternal toxicity. For the parent compound, belumosudil, a dose level of 15 mg base/kg/day corresponded to a maternal Cₘₐₓ of 1320 ng/mL at the end of the dosing regimen (GD 17), and a maternal AUC₀₋₂₄ of 11900 ng·hr/mL; for KD025 m1, maternal Cₘₐₓ was 93.3 ng/mL and AUC₀₋₂₄ was 948 ng hr/mL, and for KD025m2, maternal Cₘₐₓ was 55.6 ng/mL and AUC₀₋₂₄ was 572 ng·hr/mL.

### Example 4: An Embryo-Fetal Developmental Toxicity Study in New Zealand White Rabbits with a Toxicokinetic Evaluation

The objective of this study was to determine the embryo-fetal developmental toxicity, including the teratogenic potential, of belumosudil in New Zealand White (NZW) rabbits. This study also included a toxicokinetic evaluation to determine the exposure/toxicity relationship for the belumosudil and metabolites (KD025 m1 and KD025m2). The vehicle, 0.4% (w/w) methylcellulose (400 cps) in distilled water, or belumosudil was administered to time-mated female New Zealand White (NZW) rabbits once daily via oral gavage from Gestation Day (GD) 6 through 18. Using a standard, by weight, measured value randomization procedure, 111 female animals (weighing 2.64 to 3.56 kg, at randomization) were assigned to the control, treatment, and TK groups identified in the following Table 11.

TABLE 11
Design of Example 4 Study

| | | |
|---|---|---|
| Group | Dose Level | Number of Time-mated |
| Number | (mg base/kg/day) | Females |

Main Study

| | | |
|---|---|---|
| 1 | 0 | 23 |
| 2 | 50 | 23 |
| 3 | 125 | 23 |
| 4 | 225 | 23 |

Toxicokinetic

| | | |
|---|---|---|
| 5 | 0 | 4 |
| 6 | 50 | 5 |
| 7 | 125 | 5 |
| 8 | 225 | 5 |

Animals assigned to study had body weights within ±20% of the mean body weight. The animals were individually housed in suspended, stainless steel cages in an environmentally controlled room. Animal enrichment was provided according to SOP. Fluorescent lighting was provided for approximately 12 hours per day. The dark cycle was interrupted intermittently due to study-related activities. Temperature and humidity were monitored, recorded, and maintained to the maximum extent possible within the ranges of 61 to 72° F. and 30 to 70%, respectively. Food was offered in the morning from 08:00 to 12:00 beginning on the second day of acclimation and continuing throughout the study. During the dosing period, food was offered 1.5 hours±30 minutes prior to dosing and remained as needed until the next food offering.

### Administration

The vehicle and belumosudil were administered once per day from GD 6 to 18 at approximately the same time each day (±2 hours from the first dose on GD 6) via oral gavage. The dose levels for the treated groups were 50, 125, 225, mg base/kg/day at a dose volume of 10 mL/kg. The control group received the vehicle in the same manner as the treated groups. Additionally, TK animals received the vehicle or belumosudil in the same manner as the main study groups at the same dose levels and volume. The vehicle and belumosudil formulations were continually stirred for at least 30 minutes at room temperature prior to and throughout dose administration. Individual doses were based on the most recent body weights.

### Observations

Observations of the animals included clinical signs, body weights, food consumption, and anatomical pathology including a uterine examination. Toxicokinetic assessment was conducted for the belumosudil and metabolites (KD025 m1 and KD025m2). All fetuses were given an external, visceral, and skeletal examination.

### Cageside and Clinical Observations

All animals were observed cageside (for morbidity, mortality, injury), twice daily. Daily from GD 6 through 29 (4 hours±1 hour post dose on dosing days), each main study animal was removed from the cage and given a detailed clinical examination. On occasion, clinical observations were recorded at unscheduled intervals. The observations included, but were not limited to, evaluation of the skin, fur, eyes, ears, nose, oral cavity, thorax, abdomen, external genitalia, limbs and feet, as well as evaluation of respiration. Two animals aborted on GD 19, one each at 125 and 225 mg base/kg/day (animal numbers 251 and 282, respectively). An additional animal at 225 mg base/kg/day was found dead on GD 9. These events were considered adverse and treatment related.

A clinical finding of thin body condition was seen in the 225 mg base/kg/day group and could be correlated with decreases seen in the body weight gain and food consumption data, which were considered treatment related and adverse. Red material in the cage pan was seen for two animals which aborted at 125 and 225 mg base/kg/day, respectively, the abortions of which were considered treatment related and adverse (refer to previous section). Maternal survival and pregnancy status are summarized below in Table 12. Clinical observations are summarized below in Table 13.

TABLE 12
Main Study Maternal Survival

| Group (mg base/kg/day) | 0 | 50 | 125 | 225 |
|---|---|---|---|---|
| Number Females on Study | 23 | 23 | 23 | 23 |
| Number Not Pregnant | 0 | 1 | 3 | 0 |
| Number Aborted | 0 | 0 | 1 | 1 |
| Number Died | 0 | 0 | 0 | 1 |
| Number with All | 0 | 0 | 0 | 2 |
| Resorptions GD29 | | | | |
| Number Litters with Viable | 23 | 22 | 19 | 19 |
| Fetuses GD29 | | | | |

TABLE 13
Summary of Gestation Clinical Observations,
Days 0 to 29 (Example 4)*

| | Dose (base/kg/day) | | | |
|---|---|---|---|---|
| Observation | 0 mg | 50 mg | 125 mg | 225 mg |
| Number of Animals Observed | 23 | 23 | 23 | 23 |
| Animal Husbandry | 0/0 | 1/1 | 0/0 | 0/0 |
| Nail missing, Forefoot/left | | | | |
| Behavior/Activity | 0/0 | 0/0 | 0/0 | 4/1 |
| Activity decreased | | | | |
| Excretion | 3/2 | 0/0 | 0/0 | 0/0 |
| Feces few/absent | | | | |
| Material in pan/bedding, Red | 0/0 | 0/0 | 2/1 | 2/2 |
| External Appearance | 2/1 | 0/0 | 0/0 | 0/0 |
| Swelling, Nose/muzzle | | | | |
| Swelling, Vulva | 0/0 | 3/1 | 0/0 | 2/1 |
| Thin | 0/0 | 0/0 | 0/0 | 38/5 |
| Pelage/Skin | 0/0 | 0/0 | 7/1 | 0/0 |
| Hair discolored, Brown, | | | | |
| Anogenital region | | | | |
| Hair discolored, Red, | 0/0 | 0/0 | 1/1 | 0/0 |
| Anogenital region | | | | |
| Hair discolored, Brown, Ear/left | 0/0 | 12/1 | 0/0 | 0/0 |
| Hair discolored, Brown, | 0/0 | 12/1 | 0/0 | 0/0 |
| Ear/right | | | | |
| Hair discolored, Yellow, | 5/1 | 0/0 | 0/0 | 0/0 |
| Ear/right | | | | |
| Hair discolored, Brown, Face | 4/1 | 24/3 | 0/0 | 12/2 |
| Hair discolored, Red, Face | 1/1 | 0/0 | 2/1 | 0/0 |
| Hair discolored, Yellow, Face | 0/0 | 27/2 | 10/1 | 14/2 |
| Hair discolored, Red, | 0/0 | 1/1 | 0/0 | 0/0 |
| Forelimb/left | | | | |
| Hair discolored, Brown, Hind | 0/0 | 3/1 | 0/0 | 1/1 |
| limb/left | | | | |
| Hair discolored, Red, Thoracic | 0/0 | 1/1 | 0/0 | 0/0 |
| region | | | | |
| Hair sparse, Anogenital region | 0/0 | 0/0 | 15/2 | 0/0 |
| Hair sparse, Axillary region/left | 0/0 | 0/0 | 2/1 | 3/1 |
| Hair sparse, Axillary | 0/0 | 0/0 | 2/1 | 2/1 |
| region/right | | | | |
| Hair sparse, Cervical region | 0/0 | 0/0 | 1/1 | 0/0 |
| Hair sparse, Ear/left | 13/1 | 0/0 | 0/0 | 0/0 |
| Hair sparse, Ear/right | 4/1 | 0/0 | 0/0 | 0/0 |
| Hair sparse, Forefoot/left | 0/0 | 5/1 | 0/0 | 3/1 |
| Hair sparse, Forefoot/right | 0/0 | 4/1 | 0/0 | 5/2 |
| Hair sparse, Forelimb/left | 0/0 | 5/1 | 4/1 | 0/0 |
| Hair sparse, Hind limb/left | 0/0 | 5/1 | 0/0 | 8/1 |
| Hair sparse, Inguinal | 0/0 | 0/0 | 4/1 | 0/0 |
| region/right | | | | |
| Hair sparse, Shoulder/left | 0/0 | 0/0 | 2/1 | 0/0 |
| Hair sparse, Thoracic region | 0/0 | 0/0 | 2/1 | 1/1 |
| Hair sparse, Ventral surface | 0/0 | 0/0 | 19/1 | 0/0 |
| Scabbed area, Ear/left | 7/1 | 0/0 | 0/0 | 0/0 |
| Scabbed area, Nose/muzzle | 9/2 | 0/0 | 0/0 | 0/0 |
| Skin discolored, Pale, Entire | 0/0 | 0/0 | 0/0 | 11/1 |
| body | | | | |

*Number of times observed/Total number of animals affected.

### Body Weights.

Body weights for all animals were measured and recorded on GD 0, 6, 10, 13, 16, 19, 21, 25, and 29. Individual body weight change was calculated for the following GD intervals: 0-6, 6-10, 10-13, 13-16, 16-19, 19-21, 21-25, 25-29, 6-19, 19-29, and 0-29. Adjusted body weight (GD 29 body weight minus gravid uterine weight) and adjusted body weight change (GD 0 to 29) were also calculated. Individual body weight values were recorded for TK animals.

No adverse treatment-related effects were seen in the body weight data at 50 mg base/kg/day (mean body weights and mean body weight changes). At 125 mg base/kg/day, there were no statistically significant differences and the maximum percentage difference when mean body weight values were compared to the control group occurred on GD 19 (−5.1%).

However, there was a trend towards decreased mean body weight gain during the treatment period at 125 mg base/kg/day. The following differences were statistically significant; a mean body weight loss occurred from GD 6-10, and mean body weight gain was decreased from GD 13-16, contributing to an overall decreased mean body weight gain for the treatment period (GD 6-19). Following the treatment period at 125 mg base/kg/day, mean body weight gain was increased with statistical significance from GD 25-29, contributing to an overall mean body weight gain following treatment from GD 19-29.

For the overall study period (GD 0-29), body weight gain at 125 mg base/kg/day remained decreased compared to the respective control value; the difference from the control group was −8.6%. At 225 mg base/kg/day, mean body weights were decreased with statistical significance throughout the treatment period, and initially, following the treatment period (GD 10, 13, 16, 19, and 21); the differences from the control group ranged from −6.3% to −9.2%. When mean body weight changes were evaluated at 225 mg base/kg/day, the following differences were statistically significant; a mean body weight loss occurred from GD 6-10, and mean body weight gain was decreased from GD 10-13 and GD 13-16, contributing to an overall mean body weight loss for the treatment period (GD 6-19). However, there was a trend towards an increased mean body weight gain following treatment at 225 mg base/kg/day compared to the control group; increased mean body weight gains were statistically significant from GD 19-21 and GD 21-25 when compared to the control group, contributing to an overall increased mean body weight for the post-treatment period (GD 19-29). For the overall study period (GD 0-29), body weight gain at 225 mg base/kg/day remained decreased (statistically significant); the difference from the control group was −24.2%.

The trend towards decreased body weight gain at 125 and 225 mg base/kg/day for the overall treatment period, correlating with decreased food consumption, was considered an adverse dose-related response to belumosudil. The trend towards increased mean body weight gain and food consumption following treatment at 125 and 225 mg base/kg/day was considered indicative of a rebound/recovery effect.

Maternal body weights and body weight change are summarized below in Tables 14 and 15:

TABLE 14
Maternal Body Weights (g)/% Difference From Control

| Group (mg base/kg/day) | 0 | 50 | 125 | 225 |
|---|---|---|---|---|
| GD 10 | 3.320 | 3.316 | 3.232 | 3.110^{b}/−6.3% |
| GD 13 | 3.383 | 3.370 | 3.262 | 3.111^{b}/−8.0% |
| GD 16 | 3.463 | 3.461 | 3.282 | 3.144^{b}/−9.2% |
| GD 19 | 3.524 | 3.493 | 3.346/−5.1% | 3.227^{b}/−8.4% |
| GD 21 | 3.575 | 3.531 | 3.420 | 3.334^{a}/−6.7% |

^{a}Significantly different from control: p < 0.05
^{b}Significantly different from control: p < 0.01

TABLE 15
Maternal Body Weight Change (g)/% Difference From Control

| Group (mg base/kg/day) | 0 | 50 | 125 | 225 |
|---|---|---|---|---|
| GD 6-10 | 0.043 | 0.030 | −0.036^{b} | −0.160^{b} |
| GD 6-19 | 0.247 | 0.207 | 0.079^{b} | −0.041^{b} |
| GD 19-29 | 0.138 | 0.175 | 0.241^{b} | 0.294^{b} |
| GD 0-29 | 0.607 | 0.592 | 0.555/−8.6% | 0.460^{b}/−24.2% |

^{a}Significantly different from control: p < 0.05
^{b}Significantly different from control: p < 0.01

### Food Consumption

When food consumption at 50 mg base/kg/day was compared to the control group for the overall treatment period, GD 6-19, the percentage difference was −8.8%. At 125 mg base/kg/day, food consumption was decreased throughout the treatment period; the following differences were statistically significant; food consumption was decreased from GD 6-10, 10-13, 13-16, and 16-19, contributing to decreased food consumption for the overall treatment period (GD 6-19).

For the overall treatment period (GD 6-19) at 125 mg base/kg/day, the percentage difference from the control group was −34.1%. Following the treatment period at 125 mg base/kg/day, food consumption was increased with statistical significance from GD 21-25 and GD 25-29, contributing to an overall increase in food consumption following treatment from GD 19-29. For the overall study period (GD 0-29), food consumption at 125 mg base/kg/day remained decreased (statistical significant); the difference from the control group was −11.6%.

At 225 mg base/kg/day, food consumption was decreased throughout the treatment period; the following differences were statistically significant; food consumption was decreased from GD 6-10, 10-13, 13-16, and 16-19, contributing to decreased food consumption for the overall treatment period (GD 6-19). For the overall treatment period (GD 6-19) at 225 mg base/kg/day, the percentage difference from the control group was −49.7%. Following the treatment period at 225 mg base/kg/day, food consumption was increased with statistical significance from GD 21-25 and GD 25-29, contributing to an overall increase in food consumption following treatment from GD 19-29. For the overall study period (GD 0-29), food consumption at 225 mg base/kg/day remained decreased (statistically significant); the difference from the control group was −17.8%

The trend towards decreased food consumption for the overall treatment period, correlating with decreased body weight gain at 125 and 225 mg base/kg/day, was considered an adverse dose-related response to belumosudil treatment. The trend towards increased food consumption and mean body weight gain following treatment at 125 and 225 mg base/kg/day was considered indicative of a rebound/recovery effect.

Maternal food consumption is summarized below in Table 16:

TABLE 16
Maternal Food Consumption/% Difference From Control

| | Group (mg base/kg/day) | | | |
|---|---|---|---|---|
| | 0 | 50 | 125 | 225 |
| GD 6-10 | 159.5 | 149.4 | 117.9^{b} | 61.2^{b} |
| GD 6-19 | 156.4 | 142.7/−8.8% | 103.1^{b}/−34.1% | 78.70/−49.7% |
| GD 19-29 | 131.8 | 136.0 | 149.9^{a} | 155.3^{b} |
| GD 0-29 | 145.7 | 141.1 | 128.8^{a}/−11.6% | 119.80/−17.8% |

^{a}Significantly different from control: p < 0.05
^{b}Significantly different from control: p < 0.01

### Postmorten Uterine, Ovarian and Fetal Examinations

On GD 29, each surviving main study female was euthanized by an intravenous injection of sodium pentobarbital/euthanasia solution followed by an SOP approved method to ensure death and immediately subjected to a laparohysterectomy. The skin was reflected from a ventral midline incision to examine mammary tissue and locate any subcutaneous masses. The abdominal cavity was then opened and the uterus was exposed. The uterus was excised, and the gravid uterine weight was recorded. Beginning at the distal end of the left uterine horn, the location of viable and nonviable fetuses, early and late resorptions for each uterine horn, and the total number of implantations were recorded. The number of corpora lutea on each ovary was also recorded.

The fetuses were removed by making a dorsal incision longitudinally along both uterine horns. The embryonic membrane of each fetus was gently removed, and each fetus was pulled away from the placenta, fully extending the umbilical cord. The placentae were examined grossly.

Uteri from females that appeared nongravid were opened and placed in 10% ammonium sulfide solution for detection of implantation sites. If no foci were detected, the female was considered to be nonpregnant.

No adverse treatment-related effects were apparent at 50 mg base/kg/day when the intrauterine values were compared to those in the control group.

Postimplantation losses (%/litter) at 125 and 225 mg base/kg/day were 7.420 and 17.72, respectively, compared to 4.070/litter in the control group. Dose-related increases in postimplantation loss (%/litter) at 125 and 225 mg base/kg/day, with corresponding increases in the mean numbers of resorptions (early and late combined), and decreases in the mean numbers of viable fetuses/litter size, were considered treatment related and adverse.

At 225 mg base/kg/day, the mean fetal body weight for male fetuses was decreased (statistically significant), contributing to a decreased mean fetal body weight for the sexes combined. The male fetal body weight value (litter incidence) was 38.92 g compared to the control value of 42.05 g; the difference from the control group was 37448. The mean fetal body weight for sexes combined at 225 mg base/kg/day was 38.60 g, compared to the control value of 40.96 g; the difference from the concurrent control group was −5.76%. The mean fetal body weight for sexes combined in the historical data is 41.426 g. The effects on mean fetal body weights at 225 mg base/kg/day were considered treatment-related and adverse.

The intrauterine data are summarized below in Table 17. Fetal body weight data is reported below in Table 18.

TABLE 17
Summary of Uterine Examinations

| Group (mg base/kg/day) | 0 | 50 | 125 | 225 |
|---|---|---|---|---|
| Number Litters Evaluated GD 29 | 23 | 22 | 19 | 21 |
| Number with All Resorptions GD 29 | 0 | 0 | 0 | 2 |
| Number Litters with Viable Fetuses GD 29 | 23 | 22 | 19 | 19 |
| Mean Postimplantation Loss (%/Litter) | 4.07 | 2.42 | 7.42 | 17.72 |
| Mean Number of Resorptions (Combined) | 0.3 | 0.2 | 0.7 | 1.5^{a} |
| Mean Number of Early Resorptions | 0.3 | 0.2 | 0.5 | 1.4^{a} |
| Mean Live Litter Size | 8.4 | 8.5 | 8.0 | 7.4 |

^{a}Significantly different from control: p < 0.05

TABLE 18
Summary of Mean Fetal Body Weights
(g)/% Difference From Control

| Group (mg base/kg/day) | 0 | 50 | 125 | 225 |
|---|---|---|---|---|
| Fetal Body Weight Males | 42.05 | 41.64 | 41.61 | 38.92^{a}/−7.44% |
| Fetal Body Weight Females | 39.24 | 40.25 | 41.14 | 38.94 |
| Fetal Body Weight Genders | 40.96 | 40.97 | 41.09 | 38.60/−5.76% |
| Combined | | | | |

^{a}Significantly different from control: p < 0.05

A fetal malformation short tail (entire), occurred only at 225 mg base/kg/day, and was considered an adverse treatment-related response. There were five fetuses from two litters at 225 mg base/kg/day with this anomaly. The litter/fetal incidence was 10.5%/3.2% compared to the incidence in historical data of 5.0%/0.6%.

The fetal external malformations are summarized below in Tables 19 and 20:

TABLE 19
Fetal External Malformations

| Group (mg base/kg/day) | 0 | 50 | 125 | 225 |
|---|---|---|---|---|
| No. Litters | 1 (4.3) | 1 (4.5) | 0 (0.0) | 2 (10.5) |
| (No. Fetuses) | 1 (0.5) | 1 (0.5) | 0 (0.0) | 5 (3.2) |

TABLE 20
Fetal External Malformation: Short Tail

| Group (mg base/kg/day) | 0 | 50 | 125 | 225 |
|---|---|---|---|---|
| No. Litters | 0 (0.0) | 0 (0.0) | 0 (0.0) | 2 (10.5) |
| (No. Fetuses) | 0 (0.0) | 0 (0.0) | 0 (0.0) | 5 (3.2) |

The fetal variation tail bent (entire) was seen at 225 mg base/kg/day in two fetuses from one litter, in which both fetuses displayed the short tail anomaly described above, in addition to caudal vertebrae malformations seen upon skeletal examination. The litter (fetal) incidence of this variation was 5.3% (1.3%) in this study, and the incidence in the historical data is 4.3% (0.5%), respectively. The incidence was considered treatment-related and adverse, considering the correlative association with the short tail anomaly.

The overall incidence of fetal visceral variations was increased at 125 and 225 mg base/kg/day in comparison to controls, however, there was no dose-related trend and they were generally limited in occurrence, and typical of those seen in the historical data. This includes the fetal variation ureter malpositioned, which was increased with statistical significance (on a litter basis) at 125 (litter incidence of 21.1%) and 225 mg base/kg/day (litter incidence of 26.3%). However, the litter (fetal) incidence of ureter malpositioned is 42.1% (7.7%) in the historical data, and therefore, this variation was not considered an adverse treatment-related effect.

Fetal visceral observations are summarized in Table 21, and fetal skeletal malformations are reported in Table 22.

TABLE 21
Fetal Visceral Variations

| Group (mg base/kg/day) | 0 | 50 | 125 | 225 |
|---|---|---|---|---|
| No. Litters | 6 (26.1) | 4 (18.2) | 7 (36.8) | 8 (42.1) |
| (No. Fetuses) | 6 (3.1) | 6 (3.2) | 11 (7.2) | 12 (7.7) |

TABLE 22
Fetal Skeletal Malformations

| Group (mg base/kg/day) | 0 | 50 | 125 | 225 |
|---|---|---|---|---|
| No. Litters | 3 (13.0) | 6 (27.3) | 6 (31.6) | 10 (52.6) |
| (No. Fetuses) | 4 (2.1) | 8 (4.3) | 10 (6.6) | 19 (12.2) |

The overall incidence of fetal skeletal malformations was increased at 50, 125 and 225 mg base/kg/day, yet there were no dose-related trends apparent at 50 and 125 mg base/kg/day. However, there were trends at 225 mg base/kg/day in the fetal skeletal data, whereby the overall incidence of fetal skeletal malformations was increased, with increases in specific skeletal malformations which were typically not only increased when compared to the concurrent control group, but were also outside the range seen in the historical control data. Fetal skeletal findings which contributed primarily to the overall increase in anomalies at 225 mg base/kg/day consisted of thoracic cavity and thoracic vertebral malformations, including the findings rib(s) branched (statistically significant), fused, or misshapen, sternebra(e) fused and neural arches fused, misaligned, and misshapen (statistically significant). The finding rib(s) branched occurred at a litter/fetal incidence of 21.2% (2.6%); the incidence in the historical data is 5.0% (0.5%). The finding rib(s) fused occurred at a litter/fetal incidence of 15.8% (2.6%); the incidence in the historical data is 14.3% (1.7%). The finding rib(s) misshapen occurred at a litter/fetal incidence of 15.8% (1.9%); the incidence in the historical data is 5.9% (0.7%). The finding sternebra(e) fused occurred at a litter/fetal incidence of 31.6% (4.5%); the incidence in the historical data is 26.1% (3.9%).

The finding neural arches (thoracic) fused occurred at a litter/fetal incidence of 10.5% (1.9%); the incidence in the historical data is 9.5% (1.1%). The finding neural arches (thoracic) misaligned occurred at a litter/fetal incidence of 10.5% (1.3%); the incidence in the historical data is 5.3% (0.6%). The finding neural arches (thoracic) misshapen occurred at a litter/fetal incidence of 21.1% (2.6%); the incidence in the historical data is 9.5% (1.1%). These skeletal malformations observed at 225 mg base/kg/day were considered belumosudil related and adverse.

In addition, two fetal skeletal variations were increased (statistically significant) at 225 mg base/kg/day and were considered likely adverse and treatment-related. One of these findings was seen in the cervical vertebra(e), classified as centra, hemicentra, which occurred at a litter/fetal incidence of 21.1% (2.6%); the incidence in the historical data is 5.3% (0.6%). The other fetal skeletal variation which was increased at 225 mg base/kg/day was the finding sternebra(e) extra, which occurred at a litter/fetal incidence of 21.1% (4.5%); the incidence in the historical data is 5.3% (1.5%).

### Maternal Macroscopic Observations

Maternal necropsy findings for three animals that did not survive to the scheduled necropsy examination on GD 29 revealed the following. Two animals aborted on GD 19, one each at 125 and 225 mg base/kg/day (animal numbers 251 and 282, respectively). The urinary bladder and uterus of one subject (dosed at 125 mg base/kg/day), contained mild/minimal amounts of red fluid, and another animal at 225 mg base/kg/day was found dead on GD 9. A cause of death was not determined at necropsy; the adipose tissue of this animal was discolored yellow (moderate, icteric), and there were multiple red foci on the thymus (mild). The death was considered an adverse treatment-related effect.

### Toxicokinetic Analysis

Exposure to belumosudil, KD025 m1, and KD025m2 increased with the increase in belumosudil dose level from 50 to 225 mg base/kg. The increases in belumosudil, KD025 m1, and KD025m2 mean Cₘₐₓ values for pregnant rabbits were generally dose proportional on GD 6 and GD 18 and greater than dose proportional on GD 6 and GD 18 for AUC₀₋₂₄; however, variability was large for AUC₀₋₂₄ at the 225 mg base/kg dose level. AUC₀₋₂₄ values were generally similar or lower on GD18 than GD 6 indication no apparent accumulation of belumosudil, KD025 m1, and KD025m2 was observed after multiple dosing of belumosudil in pregnant rabbits.

The mean AUC₀₋₂₄ metabolite to parent ratios indicate that belumosudil is converted to KD025 m1 and KD025m2 in pregnant rabbits following oral gavage administration of belumosudil. Metabolite to Parent ratios ranged from 0.344 to 0.820 and 0.652 to 1.98 for KD025 m1 and KD025m2, respectively. The results of the TK analysis are reported below in Table 23.

TABLE 23
Summary of the Mean Belumosudil, KD025m1, and KD025m2
Cₘₐₓ and AUC₀₋₂₄ in Plasma of Pregnant Rabbits

| Inter- | | | belumosudil | | KD025m1 | | KD025m2 | |
|---|---|---|---|---|---|---|---|---|
| val | Dose | Dose Level | Cₘₐₓ | AUC₀₋₂₄ | Cₘₐₓ | AUC₀₋₂₄ | Cₘₐₓ | AUC₀₋₂₄ |
| (GD) | Group | (mg base/kg) | (ng/mL) | (ng*hr/mL) | (ng/mL) | (ng*hr/mL) | (ng/mL) | (ng*hr/mL) |
| 6 | 6 | 50 | 919 | 3870 | 750 | 2470 | 1170 | 4180 |
| | 7 | 125 | 3030 | 21400 | 1250 | 7490 | 2180 | 13600 |
| | 8 | 225 | 4140 | 55700 | 1790 | 21300 | 3190 | 42400 |
| 18 | 6 | 50 | 437 | 1590 | 569 | 1220 | 1120 | 2470 |
| | 7 | 125 | 1670 | 9290 | 1350 | 5740 | 3610 | 16700 |
| | 8 | 225 | 2580 | 23500 | 1680 | 14800 | 4620 | 41500 |

### Results

When belumosudil was administered to pregnant rabbits from Gestation Day 6 through 18 at 50, 125, and 225 mg base/kg/day, maternal and developmental toxicity occurred at 125 and 225 mg base/kg/day. One animal each aborted at 125 and 225 mg base/kg/day, and another animal at 225 mg base/kg/day was found dead.

Effects on body weight and food consumption during the treatment period at 125 and 225 mg base/kg/day were considered adverse and related to belumosudil treatment. Dose-related increases in postimplantation loss (comprised of early and late resorptions) at 125 and 225 mg base/kg/day, and decreases in viable fetuses/litter size, were considered treatment related and adverse. An effect on fetal body weight occurred only at 225 mg base/kg/day, and the fetal examination data (external and skeletal) revealed fetal developmental effects only at 225 mg base/kg/day. In conclusion, a dose level of 50 mg base/kg/day was considered the NOAEL for maternal and developmental toxicity.

For the parent compound, belumosudil, a dose level of 50 mg base/kg/day corresponded to a maternal Cₘₐₓ of 437 ng/mL at the end of the dosing regimen (GD 18), and a maternal AUC0-24 of 1590 ng·hr/mL; for KD025 m1, maternal Cmax was 569 ng/mL and AUC0-24 was 1220 ng·hr/mL, and for KD025m2, maternal Cmax was 1120 ng/mL and AUC0-24 was 2470 ng·hr/mL.

### Example 5: A Combination Study of Fertility and Early Embryonic Development to Implantation in Sprague-Dawley Rats

### Study Objectives and Design

This study was conducted to determine the effect of belumosudil on female estrous cycle, tubal transport, implantation and development of the embryo, and detection of functional effects on male fertility. This study design used naïve animals of both sexes, incorporated treatment of both sexes, and provided for a recovery period for the treated males based on study outcome. This study also included a toxicokinetic evaluation to determine the exposure/toxicity relationship.

Male and female Sprague-Dawley (SD) rats were obtained from Charles River Laboratories, Raleigh, North Carolina (approximately 7 to 10 weeks of age). Using a standard, by weight, randomization procedure, 273 male and 273 female animals (weighing 229 to 497 g and 169 to 242 g, respectively, at randomization) were assigned to control, treatment, and toxicokinetic (TK) groups. The group design of this study is shown in Table 24. The dose volume for each group was 10 mL/kg providing a dose concentration in mg/base/mL of one tenth the dose level). (In Table 24, T=Treated; U=Untreated).

TABLE 24
Study Design of Combination Fertility and Early Embryonic Development

| | | Number of Animals | | | | | | |
|---|---|---|---|---|---|---|---|---|
| | | Main Study | | | Recovery | | Toxicokinetics | |
| | Dose Level | Males | Females | Females | Males | Females | Males | Females |
| Group | (mg base/kg/day) | (T) | (T) | (U) | (T) | (U) | (T) | (T) |
| 1 | 0 | | 25^{a} | | | | | |
| 2 | 50 | | 25^{a} | | | | | |
| 3 | 150 | | 25^{a} | | | | | |
| 4 | 275 | | 25^{a} | | | | | |
| 5 | 0 | 25^{b} | | 25 | | | | |
| 6 | 50 | 25^{b} | | 25 | | | | |
| 7 | 150 | 25^{b} | | 25 | | | | |
| 8 | 275 | 25^{b} | | 25 | | | | |
| 9 | 0 | | | | 10^{c} | 10^{c} | | |
| 10 | 50 | | | | 10^{c} | 10^{c} | | |
| 11 | 150 | | | | 10^{c} | 10^{c} | | |
| 12 | 275 | | | | | | | |
| 13 | 0 | | | | 10^{c} | 10^{c} | 3^{d} | 3^{e} |
| 14 | 50 | | | | | | 10^{d} | 10^{e} |
| 15 | 150 | | | | | | 10^{d} | 10^{e} |
| 16 | 275 | | | | | | 10^{d} | 10^{e} |

| | | | | | | | |
|---|---|---|---|---|---|---|---|
| Total number of animals: | 100 | 100 | 100 | 40 | 40 | 33 | 33 |

Females treated 14 days prior to pairing with untreated males; males remain naïve until the end of the first mating period; males ordered to arrive at 7 weeks of age and allowed to acclimate for at least one week.
^{b}Males begin dosing after pairing with the treated females (first mating period); dosing continues for 70 days prior to pairing with untreated females (second mating period) until the GD13 uterine exams.
^{c}Males assigned to the recovery phase dosed for 70 days and allowed to recover for 70 days prior to pairing with untreated females (1:1); pairing with naïve females based on study outcome.
^{d}One cohort of 3 TK control males bled at one time point on study day 1 and 70; 2 cohorts of 5 TK males bled at alternating times on study Day 1 (6 time points) and 70 (7 time points)
^{e}One cohort of 3 TK control females bled at one time point on study Day 1 and 14; 2 cohorts of 5 TK females bled at alternating times on study Day 1 (6 time points) and 14 (7 time points)

All animals were given a detailed clinical examination prior to selection and body weights were recorded at receipt and prior to selection. Additionally, the males assigned to Groups 5-8 were given a detailed clinical observation weekly and body weights were recorded weekly during the acclimation period.

The animals were individually housed in solid bottom cages with nonaromatic bedding in an environmentally controlled room, except during pairing. During pairing, the rats were cohabited (one male and one female from the corresponding group) in the cage of the male. During acclimation periods, animals were observed twice daily with respect to general health and any signs of disease.

### Administration

The vehicle and belumosudil were administered once daily in the morning at approximately the same time each day (±2 hours from the Day 1 dose) to all treated animals via oral gavage at dose levels of 0, 50, 150, and 275 mg base/kg/day and a dose volume of 10 mL/kg. The high dose level (275 mg base/kg/day) provided an opportunity to have some mild toxicity and allowed for the evaluation of the fertility and early embryonic developmental toxicity at exposure levels that were approximately 4-fold higher than the anticipated highest steady state clinical exposures. The low (50 mg base/kg/day) and middle (150 mg base/kg/day) dose levels selected were intended to demonstrate a dose-dependent response and expected to be clinically relevant (approximately 0.5 to 1- and 2-fold of the anticipated highest steady state clinical exposures, respectively).

Dosing began at 11 weeks of age and 70 days prior to pairing (with untreated females) for the treated males, and 14 days prior to pairing (with untreated males) for the treated females. Dosing of the males continued through the mating and postmating period to euthanasia, while dosing of the females continued through the mating period to GD 7. Females with no evidence of mating were dosed for 7 days following completion of the mating period. Males designated for the recovery phase were dosed for 70 days, and then received a 77 day recovery period.

Additionally, TK animals received the vehicle or belumosudil in the same manner as the main study groups at the same dose levels and volume. TK animals were dosed for 70 and 14 days for the males and females, respectively. Dosing of TK males and females was initiated simultaneously, continuing to euthanasia. Refrigerated daily aliquots of dosing formulations were brought to room temperature while continuously stirring for at least 30 minutes prior to dosing. The vehicle and belumosudil formulations were continually stirred prior to and throughout dose administration. Individual doses were based on the most recent body weights.

### Observations and Analysis

Animals were observed cageside, for detailed clinical changes, body weights, food consumption, estrous cycle determinations, plasma analysis, sperm analysis, toxicokinetic analysis, and anatomical pathology including uterine and ovarian examinations.

Cageside observations were made at least twice daily. All animals were observed for morbidity, mortality, injury, and availability of food and water.

Detailed clinical observations were made during treatment phases: for males, twice weekly during treatment (4 hours+/−1 hour on dosing days), and for females, daily during treatment (4 hours+/−1 hour on dosing days). During non-treatment phases (males and females), detailed clinical observations occurred weekly and also prior to the scheduled uterine examination (females) on GD 13. The observations included, but were not limited to, evaluation of the skin, fur, eyes, ears, nose, oral cavity, thorax, abdomen, external genitalia, limbs and feet, as well as evaluation of respiration.

For each endpoint, treatment groups were compared to the control group using the analysis outlined in Table 25. Data for some endpoints, as indicated, were transformed by an arcsin-square root transformation prior to conducting the specified analysis.

TABLE 25
Statistical Analysis

| Endpoints | Type of Analysis |
|---|---|
| Parental In-life Data | |
| Body Weights: premating, gestation, | Group Pair-wise Comparisons |
| postmating (males) | |
| Body Weight Change: between each | Group Pair-wise Comparisons |
| weighing interval, over entire premating | |
| period (males and females), gestation, | |
| postmating (males) | |
| Food Consumption: premating, gestation, | Group Pair-wise Comparisons |
| postmating (males) | |
| Fertility Indices | |
| Copulatory Interval | |
| Fertility Index (male and female) | Group Pair-wise Comparisons |
| Mating Index (male and female) | Fisher's Exact Test |
| Fecundity Index (male and female) | Fisher's Exact Test |
| Estrous Cycle (mean cycle time and number | Fisher's Exact Test |
| cycles/period) | Group Pair-wise Comparisons |
| Pathology | |
| Male Reproductive Organ Weights (absolute | Group Pair-wise Comparisons |
| weights and relative to body weights) | |
| Female Reproductive Organ Weights | Group Pair-wise Comparisons |
| (absolute weights) | |
| Sperm Analysis (% abnormal and | Arcsin-Square-Root Transformation |
| % motility) | |
| Sperm Analysis (concentration) | Group Pair-wise Comparisons |
| Uterine Examination | |
| Total Number of Corpora Lutea/dam Total | Group Pair-wise Comparisons Group Pair- |
| Number Implantation/dam Viable | wise Comparisons Group Pair-wise |
| Embryos/dam | Comparisons Group Pair-wise Comparisons |
| Number Resorptions/dam | Arcsin-Square-Root Transformation Arcsin- |
| % Preimplantation Loss | Square-Root Transformation |
| % Postimplantation Loss | |

### Toxicokinetic Analysis

A total of 402 belumosudil, 402 KD025 m1 and 402 KD025m2 samples were analyzed using protein precipitation followed by analysis using high performance liquid chromatography followed by tandem mass spectrometric detection (LC-MS/MS). Exposure to belumosudil increased with the increase in dose level from 50 to 275 mg base/kg/day.

The increases in Cₘₐₓ and AUC₀₋₂₄ values were generally less than dose proportional on Day 1 for females and roughly dose proportional on Day 1 for males and Days 14 and 70 for females and males, respectively. Sex differences in belumosudil Cₘₐₓ and AUC₀₋₂₄ values were less than 2-fold with the exception of Group 14 (50 mg base/kg/day) on Day 1 where females were approximately 2.10 and 2.44-fold higher than males, respectively. Accumulation ratio values for AUC₀₋₂₄ ranged from 0.533 to 1.72 for females on Day 14 and from 0.859 to 1.98 for males on Day 70. Data is summarized in Table 26.

TABLE 26
Summary of Belumosudil Cₘₐₓ, AUC₀₋₂₄, and AR in Rat Plasma

| Inter- | | | | | | |
|---|---|---|---|---|---|---|
| val | Dose | Dose Level | | Cₘₐₓ | AUC₀₋₂₄ | AR |
| (Day) | Group | (mg base/kg) | Sex | (ng/mL) | (hg hr/mL) | AUC₀₋₂₄ |
| 1 | 14 | 50 | M | 2170 | 20100 | NA |
| | | | F | 4540 | 49200 | NA |
| | 15 | 150 | M | 5370 | 81600 | NA |
| | | | F | 5670 | 95700 | NA |
| | 16 | 275 | M | 7200 | 96400 | NA |
| | | | F | 7050 | 122000 | NA |
| 14 | 14 | 50 | F | 3200 | 26200 | 0.533 |
| | 15 | 150 | F | 9860 | 99500 | 1.04 |
| | 16 | 275 | F | 14900 | 209000 | 1.72 |
| 70 | 14 | 50 | M | 2480 | 21600 | 1.07 |
| | 15 | 150 | M | 10100 | 70100 | 0.859 |
| | 16 | 275 | M | 20400 | 191000 | 1.98 |

AR—Accumulation Ratio
NA—Not Applicable
M—Male
F—Female

Exposure to KD025 m1 increased with the increase in belumosudil dose level from 50 to 275 mg base/kg/day. The increases in Cₘₐₓ and AUC₀₋₂₄ values for males were less than dose proportional between the 50 to 150 mg base/kg/day dose levels and roughly dose proportional between the 150 to 275 mg base/kg/day dose levels.

The increases in Cₘₐₓ and AUC₀₋₂₄ values for females were roughly dose proportional. Sex differences in KD025 m1 Cₘₐₓ and AUC₀₋₂₄ values were less than 2-fold with the exception of Group 14 (50 mg base/kg/day) where males were approximately 2.10 to 2.62-fold higher than females. Accumulation ratio values for AUC₀₋₂₄ ranged from 0.738 to 1.49 for females on Day 14 and from 0.634 to 1.24 for males on Day 70. The metabolite to parent ratios ranged from 0.102 to 0.520 for AUC₀₋₂₄. Data is summarized in Table 27.

TABLE 27
Summary of KD025m1 Cₘₐₓ, AUC₀₋₂₄, and AR in Rat Plasma

| Inter- | | | | | | |
|---|---|---|---|---|---|---|
| val | Dose | Dose Level | | Cₘₐₓ | AUC₀₋₂₄ | AR |
| (Day) | Group | (mg base/kg) | Sex | (ng/mL) | (hg hr/mL) | AUC₀₋₂₄ |
| 1 | 14 | 50 | M | 934 | 10500 | NA |
| | | | F | 369 | 5000 | NA |
| | 15 | 150 | M | 1420 | 25600 | NA |
| | | | F | 1050 | 16100 | NA |
| | 16 | 275 | M | 2820 | 35600 | NA |
| | | | F | 2020 | 24900 | NA |
| 14 | 14 | 50 | F | 294 | 3690 | 0.738 |
| | 15 | 150 | F | 1260 | 18200 | 1.13 |
| | 16 | 275 | F | 2290 | 37000 | 1.49 |
| 70 | 14 | 50 | M | 770 | 8460 | 0.807 |
| | 15 | 150 | M | 1800 | 16200 | 0.634 |
| | 16 | 275 | M | 3350 | 44200 | 1.24 |

AR—Accumulation Ratio
NA—Not Applicable
M—Male
F—Female

Exposure to KD025m2 increased with the increase in belumosudil dose level from 50 to 275 mg base/kg/day. The increases in Cₘₐₓ and AUC₀₋₂₄ values for males were less than dose proportional between the 50 to 150 mg base/kg/day dose levels and roughly dose proportional between the 150 to 275 mg base/kg/day dose levels.

The increases in Cₘₐₓ and AUC₀₋₂₄ values for females were roughly dose proportional on Day 1 and greater than dose proportional on Day 14. Sex differences in KD025m2 Cₘₐₓ and AUC₀₋₂₄ values were generally less than 2-fold with the exception of Group 14 (50 mg base/kg/day) where males were approximately 2.58 to 3.70-fold higher than females. No noteworthy accumulation (<2-fold) of KD025m2 was observed after multiple doses of belumosudil in rats. Accumulation ratio values for AUC₀₋₂₄ ranged from 0.657 to 1.80 for females on Day 14 and from 0.701 to 1.35 for males on Day 70. The metabolite to parent ratios ranged from 0.0429 to 0.270 for AUC₀₋₂₄. Data is summarized in Table 28.

TABLE 28
Summary of KD025m2 Cₘₐₓ, AUC₀₋₂₄, and AR in Rat Plasma

| Inter- | | | | | | |
|---|---|---|---|---|---|---|
| val | Dose | Dose Level | | Cₘₐₓ | AUC₀₋₂₄ | AR |
| (Day) | Group | (mg base/kg) | Sex | (ng/mL) | (hg hr/mL) | AUC₀₋₂₄ |
| 1 | 14 | 50 | M | 501 | 5440 | NA |
| | | | F | 135 | 2110 | NA |
| | 15 | 150 | M | 686 | 13600 | NA |
| | | | F | 398 | 6860 | NA |
| | 16 | 275 | M | 1680 | 21500 | NA |
| | | | F | 925 | 11500 | NA |
| 14 | 14 | 50 | F | 125 | 1390 | 0.657 |
| | 15 | 150 | F | 523 | 7110 | 1.04 |
| | 16 | 275 | F | 1480 | 20700 | 1.80 |
| 70 | 14 | 50 | M | 410 | 4080 | 0.750 |
| | 15 | 150 | M | 1070 | 9520 | 0.701 |
| | 16 | 275 | M | 1900 | 28900 | 1.35 |

AR—Accumulation Ratio
NA—Not Applicable
M—Male
F—Female

In this fertility and early embryonic developmental toxicity study with belumosudil, effects were observed in treated males and females at 150 and 275 mg base/kg/day.

In treated females, at 275 mg base/kg/day, adverse clinical observations of abnormal feces (few/absent or discolored) and thin body condition were observed.

In addition, females at 150 and 275 mg base/kg/day had lower mean body weights, mean body weight change and reduced mean food consumption throughout the treatment period and were considered belumosudil-related and adverse. Lower mean body weight and/or body weight change and reduced food consumption were also observed at 50 mg base/kg/day in females. These differences at 50 mg base/kg/day, while potentially belumosudil-related, were sporadic in nature, slight in magnitude and not considered adverse.

At 275 mg base/kg/day, in treated females, a belumosudil-related increase in mean postimplantation loss and mean number of resorptions was observed and correlated with lower mean number of viable embryos. Ovarian and uterine parameters (corpora lutea count, number of implantation sites, viable embryos, and resorptions, and pre-and postimplantation loss) at 50 and 150 mg base/kg/day were unaffected by treatment with belumosudil. Reproductive and fertility indices were unaffected in the treated females at all dose levels evaluated. No belumosudil-related macroscopic findings or organ weight differences were observed in the treated females.

In treated males, at 275 mg base/kg/day, adverse clinical observations of abnormal feces (few/absent) and thin body condition were observed. In males, salivation was observed at 275 mg base/kg/day and at 150 mg base/kg/day and while potentially belumosudil-related, the finding was not dose responsive, was sporadic in nature and therefore not considered adverse. In addition, males at 150 and 275 mg base/kg/day had lower mean body weights, mean body weight change and reduced mean food consumption throughout the treatment period and were considered belumosudil-related and adverse.

The majority of these findings at 150 and 275 mg base/kg/day diminished in frequency during the recovery period indicating reversibility of toxicity. Lower mean body weight and/or body weight change and reduced food consumption were also observed at 50 mg base/kg/day in males. These differences at 50 mg base/kg/day, while potentially belumosudil-related, were sporadic in nature, slight in magnitude and not considered adverse.

At 275 mg base/kg/day, the treated male fertility and fecundity indices were low (72% and 75%, respectively) and considered belumosudil-related and adverse. No effects were seen in reproductive and fertility indices (mating, fertility, and fecundity parameters) at 275 mg base/kg/day at the recovery phase. Reproductive and fertility indices at 50 and 150 mg base/kg/day were unaffected by treatment with belumosudil.

At 275 mg base/kg/day, in the untreated females (25 untreated females mated to treated males), there were 6 non-pregnant untreated females, a decrease in mean number of implantation sites and mean number of viable embryos. These differences in pregnancy outcome and uterine parameters at 275 mg base/kg/day were considered related to the abnormal sperm evaluations (low motility, low mean number of sperm, and increased percentage of abnormal sperm) observed in the treated males and were considered belumosudil-related and adverse. In treated males at 275 mg/kg/day, during the recovery period, reproductive, fertility, and sperm parameters were unaffected. Male reproductive and fertility parameters were unaffected at 50 and 150 mg base/kg/day.

At terminal and recovery necropsy, belumosudil-related macroscopic observations of small testes (left and right) and epididymides (left, right, and right cauda) were observed at 275 mg/kg/dose and correlated with decreased mean organ weights (absolute). Additionally at 275 mg/kg/day, microscopic observations of the testes (minimal to severe degeneration/atrophy) and epididymides (minimal to mild luminal cellular debris) were noted in approximately 64% and 68% of terminal males, respectively; whereas at recovery necropsy, the percentage of treated males observed with these findings was approximately 30% and 40%, respectively. Macroscopic findings, organ weights, and microscopic findings were unaffected by treatment at 50 and 150 mg base/kg/day.

Exposure, as assessed by belumosudil and its metabolites, KD025 m1, and KD025m2, Cₘₐₓ and AUC₀₋₂₄ values, increased with an increase in belumosudil dose level from 50 to 275 mg base/kg/day. The increases in belumosudil Cₘₐₓ and AUC₀₋₂₄ values were generally less than dose proportional on Day 1 for females and roughly dose proportional on Day 1 for males and Days 14 and 70 for males and females.

The increases in KD025 m1 and KD025m2 Cₘₐₓ and AUC₀₋₂₄ values for males were less than dose proportional between the 50 to 150 mg base/kg/day dose levels and roughly dose proportional between the 150 to 275 mg base/kg/day dose levels. The increases in KD025 m1 and KD025m2 Cₘₐₓ and AUC₀₋₂₄ values for females were roughly dose proportional, with the exception of KD025m2 on Day 14 where the increases were greater than dose proportional.

Sex differences in belumosudil Cₘₐₓ and AUC₀₋₂₄ values were less than 2-fold with the exception of Group 14 (50 mg base/kg/day) on Day 1 where females were approximately 2.10 and 2.44-fold higher than males, respectively.

Sex differences in KD025 m1 and KD025m2 Cₘₐₓ and AUC₀₋₂₄ values were generally less than 2-fold with the exception of Group 14 (50 mg base/kg/day) where males were approximately 2.10 to 3.70-fold higher than females.

For KD025 m1, the metabolite to parent ratios ranged from 0.102 to 0.520 for AUC₀₋₂₄. For KD025m2, the metabolite to parent ratios ranged from 0.0429 to 0.270 for AUC₀₋₂₄.

Based on these results, the NOAEL for general toxicity endpoints was considered to be 50 mg base/kg/day for male and female rats [AUC₀₋₂₄ of 21600 ng-hr/mL (males) and 26200 ng-hr/mL (females) and Cₘₐₓ of 2480 ng/mL (males) and 3200 ng/mL (females)].

The NOAEL was considered to be 150 mg base/kg/day and 275 mg base/kg/day for male and female reproductive performance and fertility, respectively [AUC₀₋₂₄ of 70100 ng-hr/mL and Cₘₐₓ of 10100 ng/mL (males) and AUC₀₋₂₄ of 209000 ng-hr/mL and Cₘₐₓ of 14900 ng/mL (females)].

In treated females, the NOAEL for ovarian and uterine parameters was considered to be 150 mg base/kg/day (AUC₀₋₂₄ of 99500 ng-hr/mL and Cₘₐₓ of 9860 ng/mL).

### Example 6: United States REZUROCK™ (belumosudil) FDA Label

### Indications and Usage

REZUROCK is a kinase inhibitor indicated for the treatment of adult and pediatric patients 12 years and older with chronic graft-versus-host disease (chronic GVHD) after failure of at least two prior lines of systemic therapy. (1)

### Dosage and Administration

Recommended Dosage: 200 mg taken orally once daily with food. (2.1)

### Dosage Forms and Strengths

Tablet: 200 mg. (3)

### Contraindications

None. (4)

### Warnings and Precautions

Embryo-Fetal Toxicity: Can cause fetal harm. Advise females of reproductive potential of the potential risk to a fetus and to use effective contraception. (5.1, 8.1, 8.3)

### Drug Interactions

Strong CYP3A Inducers: Increase REZUROCK dosage to 200 mg twice daily. (7.1)

Proton Pump Inhibitors: Increase REZUROCK dosage to 200 mg twice daily. (7.1)

### Adverse Reactions

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension. (6.1)

### Use in Specific Populations

Lactation: Advise not to breastfeed. (8.2)

See 17 for PATIENT COUNSELING INFORMATION and FDA-approved patient labeling.

Full Prescribing Information

1 Indications and Usage

REZUROCK is indicated for the treatment of adult and pediatric patients 12 years and older with chronic graft-versus-host disease (chronic GVHD) after failure of at least two prior lines of systemic therapy.

2 Dosage and Administration

2.1 Recommended Dosage

The recommended dose of REZUROCK is 200 mg given orally once daily until progression of chronic GVHD that requires new systemic therapy.

Instruct the patient on the following:
Swallow REZUROCK tablets whole. Do not cut, crush, or chew tablets.
Take REZUROCK with a meal at approximately the same time each day [see Clinical Pharmacology (12.3)].
If a dose of REZUROCK is missed, instruct the patient to not take extra doses to make up the missed dose.

Treatment with REZUROCK has not been studied in patients with pre-existing severe renal or hepatic impairment. For patients with pre-existing severe renal or hepatic impairment, consider the risks and potential benefits before initiating treatment with REZUROCK [see Clinical Pharmacology (12.3)].

2.2 Dose Modifications for Adverse Reactions

Monitor total bilirubin, aspartate aminotransferase (AST), and alanine aminotransferase (ALT) at least monthly. Modify the REZUROCK dosage for adverse reactions as per Table 29.

TABLE 29
Recommended Dosage Modifications for REZUROCK for Adverse Reactions

| | | REZUROCK Dosage |
|---|---|---|
| Adverse Reaction | Severity* | Modifications |
| Hepatotoxicity [see | Grade 3 AST or ALT | Hold REZUROCK until recovery of |
| Adverse Reactions | (5x to 20x ULN) or | bilirubin, AST and ALT to Grade |
| (6.1)] | Grade 2 bilirubin | 0-1, then resume REZUROCK at the |
| | (1.5x to 3x ULN) | recommended dose. |
| | Grade 4 AST or ALT | Discontinue |
| | (more than 20x ULN) or | REZUROCK |
| | Grade ≥3 bilirubin | permanently. |
| | (more than 3x ULN) | |
| Other adverse | Grade 3 | Hold REZUROCK until recovery |
| reactions [see | | to Grade 0-1, then resume |
| Adverse Reactions | | REZUROCK at the recommended |
| (6.1)] | | dose level. |
| | Grade 4 | Discontinue |
| | | REZUROCK |
| | | permanently. |

*Based on CTCAE v 4.03

2.3 Dosage Modification Due to Drug Interactions

Strong CYP3A Inducers

Increase the dosage of REZUROCK to 200 mg twice daily when coadministered with strong CYP3A inducers [see Drug Interactions (7.1)].

Proton Pump Inhibitors

Increase the dosage of REZUROCK to 200 mg twice daily when coadministered with proton pump inhibitors [see Drug Interactions (7.1)].

3 Dosage Forms and Strengths

Each 200 mg tablet is a pale yellow film-coated oblong tablet debossed with “KDM” on one side and “200” on the other side.

4 Contraindications

None.

5 Warnings and Precautions

5.1 Embryo-Fetal Toxicity

Based on findings in animals and its mechanism of action, REZUROCK can cause fetal harm when administered to a pregnant woman. In animal reproduction studies, administration of belumosudil to pregnant rats and rabbits during the period organogenesis caused adverse developmental outcomes including embryo-fetal mortality and malformations at maternal exposures (AUC) less than those in patients at the recommended dose. Advise pregnant women of the potential risk to a fetus. Advise females of reproductive potential and males with female partners of reproductive potential to use effective contraception during treatment with REZUROCK and for at least one week after the last dose [see Use in Specific Populations (8.1, 8.3), Nonclinical Toxicology (13.1)].

6 Adverse Reactions

6.1 Clinical Trial Experience

Because clinical trials are conducted under widely variable conditions, adverse reaction rates observed in clinical trials of a drug cannot be directly compared with rates of clinical trials of another drug and may not reflect the rates observed in practice.

Chronic Graft versus Host Disease

In two clinical trials (Study KD025-213 and Study KD025-208), 83 adult patients with chronic GVHD were treated with REZUROCK 200 mg once daily [see Clinical Studies (14.1)]. The median duration of treatment was 9.2 months (range 0.5 to 44.7 months).

Fatal adverse reaction was reported in one patient with severe nausea, vomiting, diarrhea and multi-organ failure.

Permanent discontinuation of REZUROCK due to adverse reactions occurred in 18% of patients. The adverse reactions which resulted in permanent discontinuation of REZUROCK in >3% of patients included nausea (4%). Adverse reactions leading to dose interruption occurred in 29% of patients. The adverse reactions leading to dose interruption in ≥2% were infections (11%), diarrhea (4%), and asthenia, dyspnea, hemorrhage, hypotension, liver function test abnormal, nausea, pyrexia, edema, and renal failure with (2% each).

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension.

Table 30 summarizes the nonlaboratory adverse reactions.

TABLE 30
Nonlaboratory Adverse Reactions in ≥10% Patients
with Chronic GVHD Treated with REZUROCK

REZUROCK
200 mg once
daily (N = 83)

| | | |
|---|---|---|
| | All Grades | Grades 3-4 |
| Adverse Reaction | (%) | (%) |

Infections and infestations

| | | |
|---|---|---|
| Infection (pathogen not | 53 | 16 |
| specified)^{a} | | |
| Viral infection^{b} | 19 | 4 |
| Bacterial infection^{c} | 16 | 4 |

General disorders and administration site conditions

| | | |
|---|---|---|
| Asthenia^{d} | 46 | 4 |
| Edema^{e} | 27 | 1 |
| Pyrexia | 18 | 1 |

Gastrointestinal

| | | |
|---|---|---|
| Nausea^{f} | 42 | 4 |
| Diarrhea | 35 | 5 |
| Abdominal pain^{g} | 22 | 1 |
| Dysphagia | 16 | 0 |

Respiratory, thoracic and mediastinal

| | | |
|---|---|---|
| Dyspnea^{h} | 33 | 5 |
| Coughⁱ | 30 | 0 |
| Nasal congestion | 12 | 0 |

Vascular

| | | |
|---|---|---|
| Hemorrhage^{j} | 23 | 5 |
| Hypertension | 21 | 7 |

Musculoskeletal and connective tissue

| | | |
|---|---|---|
| Musculoskeletal pain^{k} | 22 | 4 |
| Muscle spasm | 17 | 0 |
| Arthralgia | 15 | 2 |

Nervous system

| | | |
|---|---|---|
| Headache^{l} | 21 | 0 |

Metabolism and nutrition

| | | |
|---|---|---|
| Decreased appetite | 17 | 1 |

Skin and subcutaneous

| | | |
|---|---|---|
| Rash^{m} | 12 | 0 |
| Pruritusⁿ | 11 | 0 |

^{a}infection with an unspecified pathogen includes acute sinusitis, device related infection, ear infection, folliculitis, gastroenteritis, gastrointestinal infection, hordeolum, infectious colitis, lung infection, skin infection, tooth infection, urinary tract infection, wound infection, upper respiratory tract infection, pneumonia, conjunctivitis, sinusitis, respiratory tract infection, bronchitis, sepsis, septic shock.
^{b}includes influenza, rhinovirus infection, gastroenteritis viral, viral upper respiratory tract infection, bronchitis viral, Epstein-Barr viremia, Epstein-Barr virus infection, parainfluenzae virus infection, Varicella zoster virus infection, viral infection.
^{c}includes cellulitis, Helicobacter infection, Staphylococcal bacteremia, catheter site cellulitis, Clostridium difficile colitis, Escherichia urinary tract infection, gastroenteritis Escherichia coli, Pseudomonas infection, urinary tract infection bacterial.
^{d}includes fatigue, asthenia, malaise.
^{e}includes edema peripheral, generalized edema, face edema, localized edema, edema.
^{f}includes nausea, vomiting.
^{g}includes abdominal pain, abdominal pain upper, abdominal pain lower.
^{h}includes dyspnea, dyspnea exertional, apnea, orthopnea, sleep apnea syndrome.
ⁱincludes cough, productive cough.
^{j}includes contusion, hematoma, epistaxis, increased tendency to bruise, conjunctival hemorrhage, hematochezia, mouth hemorrhage, catheter site hemorrhage, hematuria, hemothorax, purpura.
^{k}includes pain in extremity, back pain, flank pain, limb discomfort, musculoskeletal chest pain, neck pain, musculoskeletal pain.
ⁱincludes headache, migraine.
^{m}includes rash, rash maculo-papular, rash erythematous, rash generalized, dermatitis exfoliative.
ⁿincludes pruritus, pruritus generalized.

Table 31 summarizes the laboratory abnormalities in REZUROCK.

TABLE 31
Selected Laboratory Abnormalities in Patients
with Chronic GVHD Treated with REZUROCK

| | REZUROCK | | |
|---|---|---|---|
| | 200 mg once daily | | |
| | Grade 0-1 | Grade 2-4 | Grade 3-4 |
| | Baseline | Max Post | Max Post |
| Parameter | (N) | (%) | (%) |
| Chemistry | | | |
| Phosphate Decreased | 76 | 28 | 7 |
| Gamma Glutamyl | 47 | 21 | 11 |
| Transferase Increased | | | |
| Calcium Decreased | 82 | 12 | 1 |
| Alkaline Phosphatase | 80 | 9 | 0 |
| Increased | | | |
| Potassium Increased | 82 | 7 | 1 |
| Alanine Aminotransferase | 83 | 7 | 2 |
| Increased | | | |
| Creatinine Increased | 83 | 4 | 0 |
| Hematology | | | |
| Lymphocytes Decreased | 62 | 29 | 13 |
| Hemoglobin Decreased | 79 | 11 | 1 |
| Platelets Decreased | 82 | 10 | 5 |
| Neutrophil Count Decreased | 83 | 8 | 4 |

7 Drug Interactions

7.1 Effect of Other Drugs on REZUROCK

Strong CYP3A Inducers

Coadministration of REZUROCK with strong CYP3A inducers decreases belumosudil exposure [see Clinical Pharmacology (12.3)], which may reduce the efficacy of REZUROCK. Increase the dosage of REZUROCK when coadministered with strong CYP3A inducers [see Dosage and Administration (2.3)].

Proton Pump Inhibitors

Coadministration of REZUROCK with proton pump inhibitors decreases belumosudil exposure [see Clinical Pharmacology (12.3)], which may reduce the efficacy of REZUROCK. Increase the dosage of REZUROCK when coadministered with proton pump inhibitors [see Dosage and Administration (2.3)].

8 Use in Specific Populations

8.1 Pregnancy

Risk Summary

Based on findings from animal studies and the mechanism of action [see Clinical Pharmacology (12.1)], REZUROCK can cause fetal harm when administered to pregnant women. There are no available human data on REZUROCK use in pregnant women to evaluate for a drug-associated risk. In animal reproduction studies, administration of belumosudil to pregnant rats and rabbits during the period of organogenesis resulted in adverse developmental outcomes, including alterations to growth, embryo-fetal mortality, and embryo-fetal malformations at maternal exposures (AUC) approximately ≥3- (rat) and ≥0.07 (rabbit) times the human exposure (AUC) at the recommended dose (see Animal Data). Advise pregnant women and females of reproductive potential of the potential risk to the fetus.

In the U.S. general population, the estimated background risk of major birth defects and miscarriage in clinically recognized pregnancies is 2 to 4% and 15 to 20%, respectively.

Data

Animal Data

Embryo-fetal development studies were conducted in rats with administration of belumosudil to pregnant animals during the period of organogenesis at oral doses of 25, 50, 150, and 300 mg/kg/day in a pilot study and doses of 15, 50, and 150 mg/kg/day in a pivotal study. In the pilot study, maternal toxicity and embryo-fetal developmental effects were observed. Maternal toxicity (reduced body weight gain) occurred at 150 and 300 mg/kg/day doses. Increased post-implantation loss occurred at 50 and 300 mg/kg/day. Fetal-malformations were observed at ≥50 mg/kg/day and included absence of anus and tail, omphalocele, and dome shaped head. The exposure (AUC) at 50 mg/kg/day in rats is approximately 3 times the human exposure at the recommended dose of 200 mg.

In an embryo-fetal developmental study in rabbits, pregnant animals administered oral doses of belumosudil at 50, 125, and 225 mg/kg/day during the period of organogenesis resulted in maternal toxicity and embryo-fetal developmental effects. Maternal toxicity (body weight loss and mortality) was observed at doses ≥125 mg/kg/day. Embryo-fetal effects were observed at doses ≥50 mg/kg/day and included spontaneous abortion, increased post-implantation loss, decreased percentage of live fetuses, malformations, and decreased fetal body weight. Malformations included those in the tail (short), ribs (branched, fused or deformed), sternebrae (fused), and neural arches (fused, misaligned, and deformed). The exposure (AUC) at 50 mg/kg/day in rabbits is approximately 0.07 times the human exposure at the recommended dose of 200 mg.

8.2 Lactation

Risk Summary

There are no data available on the presence of belumosudil or its metabolites in human milk or the effects on the breastfed child, or milk production. Because of the potential for serious adverse reactions from belumosudil in the breastfed child, advise lactating women not to breastfeed during treatment with REZUROCK and for at least one week after the last dose.

8.3 Females and Males of Reproductive Potential

REZUROCK can cause fetal harm when administered to a pregnant woman [see Use in Specific Populations (8.1)].

Pregnancy Testing

Verify the pregnancy status of females of reproductive potential prior to initiating treatment with REZUROCK.

Contraception

Females

Advise females of reproductive potential to use effective contraception during treatment with REZUROCK and for at least one week after the last dose of REZUROCK. If this drug is used during pregnancy or if the patient becomes pregnant while taking this drug, the patient should be informed of the potential hazard to a fetus.

Males

Advise males with female partners of reproductive potential to use effective contraception during treatment with REZUROCK and for at least one week after the last dose of REZUROCK.

Infertility

Females

Based on findings from rats, REZUROCK may impair female fertility. The effect on fertility is reversible [see Nonclinical Toxicology (13.1)].

Males

Based on findings from rats and dogs, REZUROCK may impair male fertility. The effects on fertility are reversible [see Nonclinical Toxicology (13.1)].

8.4 Pediatric Use

The safety and effectiveness of REZUROCK have been established in pediatric patients 12 years and older. Use of REZUROCK in this age group is supported by evidence from adequate and well-controlled studies of REZUROCK in adults with additional population pharmacokinetic data demonstrating that age and body weight had no clinically meaningful effect on the pharmacokinetics of drug substance, that the exposure of drug substance is expected to be similar between adults and pediatric patients age 12 years and older, and that the course of disease is sufficiently similar in adult and pediatric patients to allow extrapolation of data in adults to pediatric patients.

The safety and effectiveness of REZUROCK in pediatric patients less than 12 years old have not been established.

8.5 Geriatric Use

Of the 186 patients with chronic GVHD in clinical studies of REZUROCK, 26% were 65 years and older. No clinically meaningful differences in safety or effectiveness of REZUROCK were observed in comparison to younger patients.

11 Description

Belumosudil is a kinase inhibitor. The active pharmaceutical ingredient is belumosudil mesylate with the molecular formula C₂₇H₂₈N₆O₅S and the molecular weight is 548.62 g/mol. The chemical name for belumosudil mesylate is 2-{3-[4-(1H-indazol-5-ylamino)-2-quinazolinyl]phenoxy}-N-(propan-2-yl) acetamide methanesulfonate (1:1). The chemical structure is as follows:

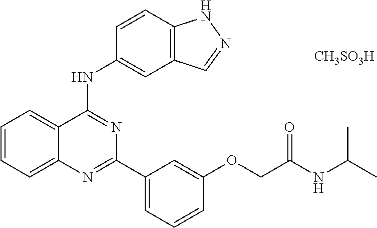 [SMILES: CC(C)NC(=O)COc1cccc(-c2nc(Nc3ccc4[nH]ncc4c3)c3ccccc3n2)c1 ; "CH3SO3H"]

Belumosudil mesylate is a yellow powder that is practically insoluble in water, slightly soluble in methanol and DMF and soluble in DMSO.

REZUROCK tablets are for oral administration. Each tablet contains 200 mg of the free base equivalent to 242.5 mg of belumosudil mesylate. The tablet also contains the following inactive ingredients: microcrystalline cellulose, hypromellose, croscarmellose sodium, colloidal silicon dioxide, and magnesium stearate.

The tablet film consists of polyvinyl alcohol, polyethylene glycol, talc, titanium dioxide and yellow iron oxide.

12 Clinical Pharmacology

12.1 Mechanism of Action

Belumosudil is an inhibitor of rho-associated, coiled-coil containing protein kinase (ROCK) which inhibits ROCK2 and ROCK1 with IC₅₀ values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of chronic GVHD.

12.2 Pharmacodynamics

Belumosudil exposure-response relationships and the time course of pharmacodynamic response are not established.

12.3 Pharmacokinetics

The following pharmacokinetic parameters are presented for chronic GVHD patients administered belumosudil 200 mg once daily, unless otherwise specified. The mean (% coefficient of variation, % CV) steady-state AUC and Cₘₐₓ of belumosudil was 22700 (48%) h·ng/mL and 2390 (44%) ng/mL, respectively. Belumosudil Cₘₐₓ and AUC increased in an approximately proportional manner over a dosage range of 200 and 400 mg (1 to 2 times once daily recommended dosage). The accumulation ratio of belumosudil was 1.4.

Absorption

Median Tₘₐₓ of belumosudil at steady state was 1.26 to 2.53 hours following administration of 200 mg once daily or twice daily in patients. The mean (% CV) bioavailability was 64% (17%) following a single belumosudil dose in healthy subjects.

Effect of Food

Belumosudil Cₘₐₓ and AUC increased 2.2 times and 2 times, respectively, following administration of a single belumosudil dose with a high-fat and high-calorie meal (800 to 1,000 calories with approximately 50% of total caloric content of the meal from fat) compared to the fasted state in healthy subjects. Median Tₘₐₓ was delayed 0.5 hours.

Distribution

The geometric mean volume of distribution after a single dose of belumosudil in healthy subjects was 184 L (geo CV % 67.7%).

Belumosudil binding to human serum albumin and human α₁-acid glycoprotein was 99.9% and 98.6%, respectively, in vitro.

Elimination

The mean (% CV) elimination half-life of belumosudil was 19 hours (39%), and clearance was 9.83 L/hours (46%) in patients.

Metabolism

Belumosudil is primarily metabolized by CYP3A4 and to a lesser extent by CYP2C8, CYP2D6, and UGT1A9, in vitro.

Excretion

Following a single oral dose of radiolabeled belumosudil in healthy subjects, 85% of radioactivity was recovered in feces (30% as unchanged) and less than 5% in urine.

Specific Populations

No clinically significant differences in belumosudil pharmacokinetics were observed with regard to age (18 to 77 years), sex, weight (38.6 to 143 kg), or mild to moderate renal impairment (eGFR≥60 and <90 mL/min/1.72 m² to eGFR≥30 and <60 mL/min/1.72 m²). The effect of severe renal impairment on the pharmacokinetics of belumosudil has not been studied.

Drug Interaction Studies

Clinical Studies and Model-Informed Approaches Effects of Other Drugs on Belumosudil

Strong Cytochrome P450 (CYP) 3A Inhibitors: There was no clinically meaningful effect on belumosudil exposure when coadministered with itraconazole in healthy subjects.

Strong CYP3A Inducers: Coadministration of rifampin decreased belumosudil Cₘₐₓ by 59% and AUC by 72% in healthy subjects.

Moderate CYP3A Inducers: Coadministration of efavirenz is predicted to decrease belumosudil Cₘₐₓ by 32% and AUC by 35% in healthy subjects.

Proton Pump Inhibitors: Coadministration of rabeprazole decreased belumosudil Cₘₐₓ by 87% and AUC by 80%, and omeprazole decreased belumosudil Cₘₐₓ by 68% and AUC by 47% in healthy subjects.

Effects of Belumosudil on Other Drugs

CYP3A Substrates: Coadministration of belumosudil is predicted to increase midazolam (a sensitive CYP3A substrate) Cₘₐₓ and AUC approximately 1.3- and 1.5-fold, respectively.

CYP2C9 Substrates: Coadministration of belumosudil is not expected to have clinically meaningful effect on the exposure of CYP2C9 substrates (such as warfarin).

CYP2C8 Substrates: Coadministration of belumosudil is not expected to have clinically meaningful effect on the exposure of CYP2C8 substrates that are not an OATP1B1 substrate.

In Vitro Studies

Transporter Systems: Belumosudil is a substrate of P-gp. Belumosudil inhibits BCRP, P-gp, and OATP1B1 at clinically relevant concentrations.

Enzymes Systems: Belumosudil is an inhibitor of CYP1A2, CYP2C19, CYP2D6, UGT1A1 and UGT1A9.

13 Nonclinical Toxicology 13.1 Carcinogenesis, Mutagenesis, Impairment of Fertility

Carcinogenicity studies have not been conducted with belumosudil.

Belumosudil was not genotoxic in an in vitro bacterial mutagenicity (Ames) assay, in vitro chromosome aberration assay in human peripheral blood lymphocytes (HPBL) or an in vivo rat bone marrow micronucleus assay.

In a combined male and female rat fertility study, belumosudil-treated male animals were mated with untreated females, or untreated males were mated with belumosudil-treated females. Belumosudil was administered orally at doses of 50, 150 or 275 mg/kg/day to male rats 70 days prior to and throughout the mating period, and to female rats 14 days prior to mating and up to Gestation Day 7. At the dose of 275 mg/kg/day, adverse findings in female rats (treated with belumosudil or untreated but mated with treated males) included increased pre- or post-implantation loss and decreased number of viable embryos. Administration of belumosudil to male rats at a dose of 275 mg/kg/day resulted in abnormal sperm findings (reduced motility, reduced count, and increased percentage of abnormal sperm), and testes/epididymis organ changes (reduced weight and degeneration).

Fertility was reduced in both treated males or females at the 275 mg/kg/day dose and reached statistical significance in males. Adverse changes in male and female reproductive organs also occurred in general toxicology studies; findings included spermatozoa degeneration at a belumosudil dose of 35 mg/kg/day in dogs and decreased follicular development in ovaries at 275 mg/kg/day in rats. Changes were partially or fully reversed during the recovery period. The exposure (AUC) at the doses of 35 mg/kg/day in dogs, and 275 mg/kg/day in rats is 0.5 times and 8-9 times, respectively, the clinical exposure at the recommended dose of 200 mg daily.

14 Clinical Studies

14.1 Chronic Graft versus Host Disease

Study KD025-213 (NCT03640481) was a randomized, open-label, multicenter study of REZUROCK for treatment of patients with chronic GV3D who had received 2 to 5 prior lines of systemic therapy and required additional treatment. Patients were excluded from the studies if platelets were <50×10⁹/L; absolute neutrophil count <1.5×10⁹/L; AST or ALT>3×ULN; total bilirubin>1.5×ULN; QTc(F)>480 ins; eGFR (30 mL/min/1.73 in²; or FE1≤39%. There were 66 patients treated with REZUROCK 200 mg taken orally once daily. Concomitant treatment with supportive care therapies for chronic GVHD was permitted. Concomitant treatment with GVHD prophylaxis and standard care systemic chronic GVHD therapies was permitted as long as the subject has been on a stable dose for at least 2 weeks prior to study. Initiation of new systemic chronic GVHD therapy while on study was not permitted.

Demographics and baseline characteristics are summarized in Table 32.

TABLE 32
Demographics and Baseline Characteristics
of Patients with Chronic GVHD

| | REZUROCK | |
|---|---|---|
| | 200 mg once | |
| | daily (N = 65) | |
| Age, Median, Years (minimum, maximum) | 53 | (21, 77) |
| Age ≥65 Years, n (%) | 17 | (26) |
| Male, n (%) | 42 | (65) |
| Race, n (%) | | |
| White | 54 | (83) |
| Black | 6 | (9) |
| Other or Not Reported | 5 | (8) |
| Median (range) time (months) from Chronic GVHD | 25.3 | (1.9, 162.4) |
| Diagnosis | | |
| ≥4 Organs Involved, n (%) | 31 | (48) |
| Median (range) Number of | 3 | (2, 6) |
| Prior Lines of Therapy | | |
| Number of Prior Lines of | | |
| Therapy, n (%) | | |
| 2 | 23 | (35) |
| 3 | 12 | (19) |
| 4 | 15 | (23) |
| ≥5 | 15 | (23) |
| Prior chronic GVHD treatment | 21 | (32) |
| with ibrutinib, n (%) | | |
| Prior chronic GVHD treatment | 20 | (31) |
| with ruxolitinib, n (%) | | |
| Refractory to Last Therapy, n (%^{a}) | 43/55 | (78) |
| Severe chronic GVHD, n (%) | 46 | (71) |
| Median (range) Global Severity Rating | 7 | (2, 9) |
| Median (range) Lee Symptom Scale | 27 | (7, 56) |
| Score at baseline | | |
| Median (range) Corticosteroid dose at | 0.19 | (0.03, 0.95) |
| baseline (PE/kg)^{b} | | |

^{a}Denominator excludes patients with unknown status
^{b}Prednisone equivalents/kilogram

The efficacy of REZUROCK was based on overall response rate (ORR) through Cycle 7 Day 1 where overall response included complete response or partial response according to the 2014 NIH Response Criteria. The ORR results are presented in Table 33. The ORR was 7500 (9500 CI: 63, 85). The median duration of response, calculated from first response to progression, death, or new systemic therapies for chronic GVHD, was 1.9 months (9500 CI: 1.2, 2.9). The median time to first response was 1.8 months (9500 CI: 1.0, 1.9). In patients who achieved response, no death or new systemic therapy initiation occurred in 620% (9500 CI: 46, 74) of patients for at least 12 months since response.

TABLE 33
Overall Response Rate through Cycle 7 Day 1 for
Patients with Chronic GVHD in Study KD025-213

| | REZUROCK |
|---|---|
| | 200 mg once daily |
| | (N = 65) |
| Overall Response Rate (ORR) | 49 (75%) |
| 95% Confidence Interval^{a} | (63%, 85%) |
| Complete Response | 4 (6%) |
| Partial Response | 45 (69%) |

^{a}Estimated using Clopper-Pearson method

ORR results were supported by exploratory analyses of patient-reported symptom bother which showed at least a 7-point decrease in the Lee Symptom Scale summary score through Cycle 7 Day 1 in 52% (95% CI: 40, 65) of patients.

16 how Supplied/Storage and Handling

REZUROCK 200 mg tablets are supplied as pale yellow film-coated oblong tablets containing 200 mg of belumosudil (equivalent to 242.5 mg belumosudil mesylate). Each tablet is debossed with “KDM” on one side and “200” on the other side and is packaged as follows:

200 mg tablets in 30 count bottle: NDC 79802-200-30

Store at room temperature, 20° C. to 25° C. (68° F. to 77° F.); excursions permitted from 15° C. and 30° C. (59° F. to 86° F.) [see USP Controlled Room Temperature].

Dispense to patient in original container only. Store in original container to protect from moisture. Replace cap securely each time after opening. Do not discard desiccant.

17 Patient Counseling Information

Advise the patient to read the FDA-approved patient labeling (Patient Information).

### Embryo-Fetal Toxicity:

Advise pregnant women and females of reproductive potential of the potential risk to a fetus. Advise females of reproductive potential to inform their healthcare provider of a known or suspected pregnancy [see Warnings and Precautions (5.1), Use in Specific Populations (8.1, 8.3)].

Advise females of reproductive potential to use effective contraceptive during treatment with REZUROCK and for at least one week after the last dose [see Warnings and Precautions (5.1)].

Advise males with female partners of reproductive potential to use effective contraceptive during treatment with REZUROCK and for at least one week after the last dose [see Use in Specific Populations (8.3)].

Lactation

Advise women not to breastfeed during treatment with REZUROCK and for at least one week after the last dose [see Use in Specific Populations (8.2)].

Infertility

Advise males and females of reproductive potential that REZUROCK may impair fertility [see Use in Specific Populations (8.3)].

Administration

Inform patients to take REZUROCK orally once daily with food according to their physician's instructions and that the oral dosage (tablets) should be swallowed whole with a glass of water without cutting, crushing or chewing the tablets approximately the same time each day [see Dosage and Administration (2.1)].

Advise patients that in the event of a missed daily dose of REZUROCK, it should be taken as soon as possible on the same day with a return to the normal schedule the following day. Patients should not take extra doses to make up the missed dose [see Dosage and Administration (2.1)].

Drug Interactions

Advise patients to inform their health care providers of all concomitant medications, including prescription medicines, over-the-counter drugs, vitamins, and herbal products [see Drug Interactions (7)].

TABLE 34
Patient Information
PATIENT INFORMATION REZUROCK (REZ-ur-ok)

(belumosudil) tabletsWhat is REZUROCK?
REZUROCK is a prescription medicine used to treat adults and children 12 years of age and
older with chronic graft- versus-host disease (chronic GVHD) after you have received at least 2
prior treatments (systemic therapy) and they did not work.
It is not known if REZUROCK is safe and effective in children less than 12 years old.
Before taking REZUROCK, tell your healthcare provider about all of your medical
conditions, including if you:
have kidney or liver problems.
are pregnant or plan to become pregnant. REZUROCK can harm your unborn baby. If
you are able to become pregnant, your healthcare provider will do a pregnancy test
before starting treatment with REZUROCK. Tell your healthcare provider if you become
pregnant or think you may be pregnant during treatment with REZUROCK.
Females who can become pregnant should use effective birth control during treatment
with REZUROCK and for at least 1 week after the last dose.
Males with female partners who can become pregnant should use effective birth
control during treatment with REZUROCK and for at least 1 week after the last dose.
are breastfeeding or plan to breastfeed. It is not known if REZUROCK passes into breast
milk. Do not breastfeed during treatment with REZUROCK and for at least 1 week after
the last dose.
Tell your healthcare provider about all the medicines you take, including prescription and
over-the-counter medicines, vitamins, and herbal supplements. REZUROCK may affect the way
other medicines work, and other medicines may affect the way REZUROCK works.
Know the medicines you take. Keep a list of them to show your healthcare provider and
pharmacist when you get a new medicine.
How should I take REZUROCK?
Take REZUROCK exactly as your healthcare provider tells you to take it.
Do not change your dose or stop taking REZUROCK without first talking to your healthcare
provider.
Take REZUROCK 1 time a day with a meal.
Take REZUROCK at about the same time each day.
Swallow REZUROCK tablets whole with a glass of water.
Do not cut, crush, or chew REZUROCK tablets.
Your healthcare provider will do blood tests to check your liver at least 1 time a
month during treatment with REZUROCK.
If you miss a dose of REZUROCK, take it as soon as you remember on the same day. Take
your next dose of REZUROCK at your regular time on the next day. Do not take extra doses
of REZUROCK to make up for a missed dose.
If you take too much REZUROCK, call your healthcare provider or go to the nearest hospital
emergency room right away.
What are the possible side effects of REZUROCK?
The most common side effects of REZUROCK include:

| | |
|---|---|
| infections | swelling |
| tiredness or weakness | bleeding |
| nausea | stomach (abdominal) pain |
| diarrhea | muscle or bone pain |
| shortness of breath | headache |
| cough | high blood pressure |

Your healthcare provider may change your dose of REZUROCK, temporarily stop, or
permanently stop treatment with REZUROCK if you have certain side effects.
REZUROCK may affect fertility in males and females. Talk to your healthcare provider if this is a
concern for you. These are not all the possible side effects of REZUROCK.
Call your doctor for medical advice about side effects. You may report side effects to FDA at 1-
800-FDA-1088. You may also report side effects to Kadmon Pharmaceuticals, LLC at 1-877-
377-7862.
How should I store REZUROCK?
Store REZUROCK at room temperature between 68° F. to 77° F. (20° C. to 25° C.).
Keep REZUROCK in its original container. The REZUROCK bottle contains a desiccant
packet to help keep your tablets dry (protect from moisture). Keep the desiccant in the
bottle.
Tightly close the REZUROCK bottle after you take your dose.
Keep REZUROCK and all medicines out of the reach of children.
General information about the safe and effective use of REZUROCK.
Medicines are sometimes prescribed for purposes other than those listed in a Patient Information
leaflet. Do not use REZUROCK for a condition for which it was not prescribed. Do not give
REZUROCK to other people, even if they have
the same symptoms that you have. It may harm them. You can ask your pharmacist or
healthcare provider for information about REZUROCK that is written for health professionals.
What are the
ingredients in
REZUROCK? Active
ingredient:
belumosudil mesylate
Inactive ingredients:
Tablet core: microcrystalline cellulose, hypromellose, croscarmellose sodium, colloidal silicon
dioxide, and magnesium stearate.
Tablet coating: polyvinyl alcohol, polyethylene glycol, talc, titanium dioxide and yellow iron oxide.

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

1-31. (canceled)

32. A method of treating a female patient of reproductive potential for chronic graft-versus-host disease (cGVHD), comprising the steps of:
(a) verifying the patient's pregnancy status;
(b) advising the patient of potential risks of taking a mesylate salt of 2-{3-[4-(1H-indazol-5-ylamino)-2-quinazolinyl]phenoxy}-N-(propan-2-yl) acetamide (Compound) to a fetus while pregnant;
(c) advising the patient to use contraception during treatment with the Compound and for at least one week after receiving the last dose of the Compound; and
(d) administering the Compound.

33. The method according to claim 32, wherein the patient is using effective contraception during treatment with the Compound and for at least one week after the last dose of the Compound.

34. The method according to claim 32, wherein the patient is lactating, further comprising the step of advising the patient not to breastfeed during treatment with the Compound and for at least one week after the last dose of the Compound.

35. The method of claim 32, wherein the patient is not lactating.

36. (canceled)

37. The method according to claim 32, wherein the Compound is administered to the patient at a dose of 200 mg once or twice daily.

38. The method according to claim 32, wherein the patient has chronic graft-versus-host disease and has failed at least two prior lines of systemic therapy for the chronic graft-versus-host disease.

39. The method according to claim 38, wherein the prior lines of systemic therapy are selected from prednisone, tacrolimus, ECP, sirolimus, ibruitinib, ruxolitinib, MMF, rituximab, MTX, cyclosporine, imatinib, ixazomib, and ofatumumab.

40. A method of treating a male patient of reproductive potential with a female partner of reproductive potential for chronic graft-versus-host disease (cGVHD) comprising the steps of:
(a) advising the patient of potential risks to a fetus if the patient impregnates a partner while receiving treatment with a mesylate salt of 2-{3-[4-(1H-indazol-5-ylamino)-2-quinazolinyl]phenoxy}-N-(propan-2-yl) acetamide (Compound);
(b) advising the patient to use contraception during treatment with the Compound and for at least one week after receiving the last dose of the Compound; and
(c) administering the Compound.

41. The method according to claim 40, wherein it is verified in step (a) that the patient is a patient of reproductive potential, further comprising the step of advising the patient to use effective contraception during treatment with the Compound and for at least one week after receiving the last dose thereof.

42. The method according to claim 40, wherein the patient is using effective contraception during treatment with the Compound and for at least one week after the last dose of the Compound.

43. (canceled)

44. The method according to claim 40, wherein the Compound is administered to the patient at a dose of 200 mg once or twice daily.

45. The method according to claim 40, wherein the patient has chronic graft-versus-host disease and has failed at least two prior lines of systemic therapy for the chronic graft-versus-host disease.

46. The method according to claim 45, wherein the prior lines of systemic therapy are selected from prednisone, tacrolimus, ECP, sirolimus, ibruitinib, ruxolitinib, MMF, rituximab, MTX, cyclosporine, imatinib, ixazomib, and ofatumumab.

47. A method of treating chronic graft-versus-host disease (cGVHD) in a patient in need thereof comprising the steps of:
(a) verifying the patient is a reproductive risk patient; and
(b) advising the patient of potential reproductive risks in receiving treatment with a mesylate salt of 2-{3-[4-(1H-indazol-5-ylamino)-2-quinazolinyl]phenoxy}-N-(propan-2-yl) acetamide (Compound); and administering the Compound.

48. The method according to claim 47, wherein the patient is a female patient.

49. The method according to claim 48, further comprising the step of verifying the pregnancy status of the patient before initiating treatment.

50. The method according to claim 48, wherein the patient becomes pregnant while receiving treatment with the Compound.

51. The method according to claim 48, wherein it is verified in step (a) that the patient is a lactating patient, further comprising the step of advising the patient not to breastfeed a child during treatment with the Compound and for at least one week after receiving the last dose thereof.

52. The method according to claim 47, wherein the patient is a male patient.

53. The method according to claim 47, comprising the step of advising the patient of fertility risks associated with treatment with the Compound.

54. The method according to claim 47, wherein the Compound is administered to the patient at a dose of 200 mg once or twice daily.

55. The method according to claim 47, wherein the patient verified in step (a) has chronic graft-versus-host disease and has failed at least two prior lines of systemic therapy for the chronic graft-versus-host disease.

56. The method according to claim 55, wherein the prior lines of systemic therapy are selected from prednisone, tacrolimus, ECP, sirolimus, ibruitinib, ruxolitinib, MMF, rituximab, MTX, cyclosporine, imatinib, ixazomib, and ofatumumab.

57. (canceled)

58. A method of treating chronic graft-versus-host disease (cGVHD), in a patient in need thereof comprising the steps of:
(a) verifying the patient is a reproductive risk patient; and
(b) advising the patient to use effective contraception; and administering a mesylate salt of 2-{3-[4-(1H-indazol-5-ylamino)-2-quinazolinyl]phenoxy}-N-(propan-2-yl) acetamide.

59. (canceled)

60. The method according to claim 58, wherein the Compound is administered to the patient at a dose of 200 mg once or twice daily.

61. The method according to claim 58, wherein the patient verified in step (a) has chronic graft-versus-host disease and has failed at least two prior lines of systemic therapy for the chronic graft-versus-host disease.
